(19) United States
(12) Patent Application Publication
(10) Pub. No.: US 2026/0253713 A1
(43) Pub. Date: Aug. 27, 2026

(54) INTELLIGENT HOSPITAL SCENARIO SIMULATION AND EMULATION

(71) Applicant: GE Precision Healthcare LLC, Waukesha, WI (US)

(72) Inventors: Md Atiur Rahman Siddique, Bothell, WA (US); Abu Mirza, Redmond, WA (US); Bree Anna Bush, Orlando, FL (US); Hariharan Balasubramanian, Redmond, WA (US); Michael Donoghue, Orlando, FL (US); Sundar Balasubramanian, Seattle, WA (US); Mark Stefan Grum, Plymouth, MI (US)

(21) Appl. No.: 19/543,265

(22) Filed: Feb. 18, 2026

Related U.S. Application Data
(60) Provisional application No. 63/762,930, filed on Feb. 25, 2025.

(51) Int. Cl. G16H 40/20

(52) U.S. Cl. CPC G16H 40/20

(57) ABSTRACT

Systems or techniques that facilitate intelligent hospital scenario simulation and emulation are provided. In various embodiments, a system can generate a configuration of resources or patient flow of one or more hospital units. In various aspects, the system can simulate a scenario in the one or more hospital units using the configuration to generate simulation data. In various instances, the system can generate action plan recommendations for operational bottlenecks detected based on the simulation data.

### CROSS REFERENCE TO RELATED APPLICATION

This application claims the benefit of and priority to U.S. Provisional Patent Application No. 63/762,930, filed on February 25, 2025, the disclosure of which is incorporated herein by reference in its entirety.

### TECHNICAL FIELD

The subject disclosure relates generally to hospital scenario modeling, and more specifically to intelligent hospital scenario simulation and emulation.

### BACKGROUND

In healthcare facilities such as hospitals and large clinics, operational efficiency is often constrained by resource availability and demand fluctuations. Existing techniques to detect and address operational bottlenecks resulting from such constraints often rely on manual inspection, retrospective analysis, and building specific simulation software.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate intelligent hospital scenario simulation and emulation are described.

According to one or more embodiments, a system is provided. The system can comprise at least one non-transitory computer-readable memory that can store computer-executable components. The system can further comprise at least one processor that can be operably coupled to the at least one non-transitory computer-readable memory and that can execute the computer-executable components stored in the at least one non-transitory computer-readable memory. In various embodiments, the computer-executable components can comprise a modeling component that can generate scenario configuration data and scenario configuration metadata representing a configuration of resources or patient flow of one or more hospital units; store the scenario configuration data in object storage; and store the scenario configuration metadata in metadata storage. In various aspects, the computer-executable components can comprise a simulation component that can retrieve the scenario configuration metadata from the metadata storage; access the scenario configuration data based on the scenario configuration metadata; simulate a scenario in the one or more hospital units using the scenario configuration data and the scenario configuration metadata representing the configuration within one or more containerized runtime instances to generate simulation data; update the scenario configuration data in the object storage based on the simulation data; and update metadata stored in the metadata storage to reflect updated scenario configuration data. In various instances, the computer-executable components can comprise an analysis component that can dynamically invoke at least one large language model (LLM) to generate, within one or more containerized runtime instances, action plan recommendations for operational bottlenecks detected based on the simulation data; and store the action plan recommendations in the object storage, and wherein the one or more containerized runtime instances can execute within a layered virtualized computing environment that provides isolation between the one or more containerized runtime instances.

According to one or more embodiments, a computer-implemented method is provided. In various embodiments, the computer-implemented method can comprise generating, by a system operatively coupled to a processor, a configuration of resources or patient flow of one or more hospital units. In various aspects, the computer-implemented method can comprise simulating, by the system, a scenario in the one or more hospital units using the configuration to generate simulation data. In various instances, the computer-implemented method can comprise generating, by the system, action plan recommendations for operational bottlenecks detected based on the simulation data.

According to one or more embodiments, a computer program product for facilitating intelligent hospital scenario simulation and emulation is provided. In various embodiments, the computer program product can comprise a non-transitory computer-readable memory having program instructions embodied therewith. In various aspects, the program instructions can be executable by a processor to cause the processor to generate, by the processor, a configuration of resources or patient flow of one or more hospital units. In various instances, the program instructions can be further executable to cause the processor to simulate, by the processor, a scenario in the one or more hospital units using the configuration to generate simulation data. In various cases, the program instructions can be further executable to cause the processor to generate, by the processor, action plan recommendations for operational bottlenecks detected based on the simulation data.

### DESCRIPTION OF THE DRAWINGS

FIG. 1 illustrates a block diagram of an example, non-limiting system that facilitates intelligent hospital scenario simulation and emulation in accordance with one or more embodiments described herein.

FIG. 2 illustrates a block diagram of an example, non-limiting system including a language embedding model and a large language model (LLM) that facilitates intelligent hospital scenario simulation and emulation in accordance with one or more embodiments described herein.

FIG. 3 illustrates an example, non-limiting block diagram showing a configuration and a plurality of resources of a hospital in accordance with one or more embodiments described herein.

FIG. 4 illustrates a block diagram of an example, non-limiting system including a display component and a graphical user interface (GUI) that facilitates intelligent hospital scenario simulation and emulation in accordance with one or more embodiments described herein.

FIG. 5 illustrates a block diagram of an example, non-limiting system including a GUI that facilitates intelligent hospital scenario simulation and emulation in accordance with one or more embodiments described herein.

FIG. 6 illustrates a flow diagram of an example, non-limiting computer-implemented method that facilitates configuration generation in accordance with one or more embodiments described herein.

FIG. 7. illustrates an example, non-limiting block diagram showing generation of simulation summaries and data visuals in accordance with one or more embodiments described herein.

FIG. 8 illustrates an example, non-limiting block diagram showing a GUI that facilitates intelligent hospital scenario simulation and emulation in accordance with one or more embodiments described herein.

FIG. 9 illustrates a flow diagram of an example, non-limiting computer-implemented method that facilitates determination of alternate configurations in accordance with one or more embodiments described herein.

FIG. 10 illustrates a block diagram of an example, non-limiting system including hospital data and text input that facilitates intelligent hospital scenario simulation and emulation in accordance with one or more embodiments described herein.

FIG. 11 illustrates a block diagram of an example, non-limiting system including a security component that facilitates intelligent hospital scenario simulation and emulation in accordance with one or more embodiments described herein.

FIG. 12 illustrates a diagram of an example, non-limiting GUI showing simulation of a scenario using a configuration in accordance with one or more embodiments described herein.

FIG. 13 illustrates a diagram of an example, non-limiting GUI showing a simulation of a scenario in accordance with one or more embodiments described herein.

FIG. 14 illustrates a diagram of an example, non-limiting GUI showing a simulation of a scenario in accordance with one or more embodiments described herein.

FIG. 15 illustrates a diagram of an example, non-limiting GUI showing simulation data and a simulation summary in accordance with one or more embodiments described herein.

FIGS. 16-26 illustrate example, non-limiting data visuals and data summaries in accordance with one or more embodiments described herein.

FIG. 27 illustrates a flow diagram of an example, non-limiting computer-implemented method that facilitates intelligent hospital scenario simulation and emulation in accordance with one or more embodiments described herein.

FIG. 28 and 29 illustrate a flow diagram of an example, non-limiting computer-implemented method that facilitates intelligent hospital scenario simulation and emulation in accordance with one or more embodiments described herein.

FIG. 30 illustrates a block diagram of an example, non-limiting distributed cloud computing architecture in which one or more embodiments described herein can be facilitated.

FIG. 31 illustrates a block diagram of an example, non-limiting system architecture in which one or more embodiments described herein can be facilitated.

FIG. 32 illustrates a block diagram of an example, non-limiting system architecture in which one or more embodiments described herein can be facilitated.

FIG. 33 illustrates a block diagram of an example, non-limiting operating environment in which one or more embodiments described herein can be facilitated.

FIG. 34 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Efficient resource management is a critical challenge in healthcare facilities, including hospitals and large clinics, where operational bottlenecks can directly impact patient care. Operational bottlenecks are points in patient care processes where limited resources restrict throughput or create delays. These facilities often face resource constraints that arise due to the complex and dynamic nature of patient demand, staffing availability, and equipment utilization. For example, limited trauma beds in emergency departments, intensive care unit (ICU) capacity shortages, or long wait times for magnetic resonance imaging (MRI) scans can cause delays in treatment, prolonged patient stays, and reduced overall efficiency of the hospital.

While healthcare administers can address short-term issues through immediate adjustments in the field, persistent inefficiencies demand a more structured and data-driven approach to decision-making when addressing operational bottlenecks. For example, healthcare administers may consider investing in a larger building to perform patient care with more beds and MRI machines. However, deciding how many beds or MRI machines demands more educated feedback as it can be a costly over-investment that may not be the most efficient management of resources. For instance, the healthcare administers may invest more money to add 100 beds when only 50 beds will be most efficient. In other instances, the healthcare administers may invest less money in MRI machines when more MRI machines may be needed to prevent long wait times for patients. In any case, it can be desirable to detect operational bottlenecks in healthcare facilities and determine efficient resource utilizations that can prevent or resolve such operational bottlenecks.

Existing techniques facilitate such detection and resolving of operational bottlenecks in a manual, retrospective fashion. In particular, when existing techniques are implemented, bottlenecks are typically identified only after they have already impacted hospital operations, relying on post-event analysis, manual audits, or historical performance reviews. This approach can delay corrective actions and limits the ability to evaluate alternative operational strategies before problems arise. In other words, such existing techniques are reactive rather than proactive, requiring significant time and human effort to analyze past data, derive insights, and implement corrective actions, rather than simulating or emulating real-time hospital conditions to prevent bottlenecks before they occur.

Moreover, even when simulation data is available, it is often complex and difficult for non-technical end users, such as clinicians, administrators, and operational managers, to interpret and use effectively. The raw output from simulations may consist of large datasets, statistical distributions, or time-series predictions that require expertise to interpret. Without clear visualization, structured reporting, or AI-assisted analysis, users can struggle to extract actionable insights from the data. As a result, critical decision-making processes may be hindered, and the full potential of simulation-driven hospital optimization may not be realized.

Another limitation of existing approaches is that they often demand healthcare facilities to develop scenario-specific simulation software for each operational scenario or configuration of interest. Such software typically involves custom modeling of hospital units, departments, patient flow, and resource allocations, which can be time-consuming, expensive, and technically demanding. Each new scenario, such as changes in patient volume, staffing schedules, or equipment availability, can involve substantial redevelopment or reconfiguration of the simulation models. Consequently, the effort and cost associated with building and maintaining scenario-specific simulation software can limit the practical adoption of simulation techniques and reduce the ability to rapidly evaluate multiple operational strategies or alternative resource allocations.

Accordingly, systems or techniques that can address one or more of these technical problems can be desirable.

As used herein:

A hospital unit (or unit) refers to a distinct clinical area responsible for specific resources, such as beds, patient monitoring devices, diagnostic imaging equipment (e.g., X-ray, CT, or MRI machines), or human resources (e.g., care nurses, technicians). Examples include surgery units, inpatient units, outpatient units, intensive care units, and rehabilitation units.

A department refers to an organizational entity comprising two or more units coordinating functional teams that orchestrate multiple aspects of patient care. For example, a neurology department can comprise surgery units, inpatient units, outpatient units, intensive care units, and rehabilitation units.

A hospital refers to an institution comprising two or more departments that focus on distinct areas of patient care. For example, a hospital can include neurology, cardiology, oncology, pediatrics, orthopedics, and emergency medicine departments.

Patient flow refers to the movement of patients through hospital units, encompassing admission, treatment, transfer, and release. Patient flow can be characterized by parameters such as patient arrival rate, treatment time, unit capacity (e.g., number of beds), transfer patterns, release patterns, or any other operational parameters, and can vary over time (e.g., times of day, days of the week, or seasons).

A scenario refers to a combined configuration of departments, units, resources, patient flow, and any other parameters of interest of a hospital. When such parameters change considerably, the resulting configuration embodies a new scenario. For example, a scenario can comprise a neurology department with its associated units, specific patient flow patterns for peak hours, and available resources such as beds and imaging equipment. Considerably changing any of these parameters, such as increasing patient arrival rates, reallocating resources, or changing the department, results in a new scenario.

An operational bottleneck (or bottlenecks) refers to any point within a hospital, department, unit, or patient care process where limited resources, staffing, infrastructure can restrict throughput, create delays, or constrain performance. Examples include constrained bed availability, limited medical equipment, or reduced staff capacity causing reduced throughput.

Simulation refers to a method of creating a virtual, simplified (through abstractions) representation of hospital operations, processes, or resource utilization to analyze, predict, or evaluate outcomes of varying conditions.

Emulation refers to a method of reproducing the behavior of hospital operations, processes, or resource interactions that mirrors real-world conditions.

Various embodiments described herein can address one or more of these technical problems. One or more embodiments described herein can include systems, computer-implemented methods, apparatus, or computer program products that can facilitate intelligent hospital scenario simulation and emulation. More specifically, various embodiments described herein can employ simulation data to determine prospective operational bottlenecks. For example, operational bottlenecks can include long patient wait times to an imaging procedure (e.g., MRI, CT, Xray, etc.), surgical procedure (e.g., due to lack of operation theatre or equipment), admission (e.g., due to lack of a clean bed), or personnel (e.g., due to unavailability of a surgeon or care nurse). In response to detecting such operational bottlenecks, the various embodiments described herein can generate action plan recommendations (e.g., improvement opportunities) that can remedy the detected operational bottlenecks, where such action plan recommendations can further include the under-use or over-use of resources (e.g., under-use of beds or equipment).

The various embodiments described herein utilize real data extracted from electronic medical record (EMR) systems of the hospital to generate resource configurations and patient flows in each hospital unit for simulating a scenario. Users can further modify the scenario or create a new scenario. In some instances, the various embodiments described herein can employ AI-based techniques to assist users in modifying or creating scenarios.

In any case, the one or more embodiments can perform a simulation of the scenario, and extract simulation data thereafter that reflects operational metrics, resource utilization, patient flow patterns, or other hospital performance metrics. Then, the simulation data can be used to create various data visualizations to present to the user.

Thus, the embodiments described herein offer full transparency to users by providing relevant what’s, why’s, and how’s when analyzing hospital operations. Particularly, the various embodiments described herein can provide AI-driven insights and action plan recommendations from scenario simulation and emulation in a manner that is easily understandable to users. Further, the various embodiments described herein can detect existing and prospective operational bottlenecks, as well as measure impacts of potential improvement investments so that users can plan for the largest return on investment. Such informed planning optimizes patient flow, optimizes resource utilization, reduces patient wait times, and helps allocate larger funds to clinical care by saving from operational budgets and, thereby, plays a pivotal role in improving care quality for all patients.

Various embodiments described herein can be considered as a computerized tool (e.g., any suitable combination of computer-executable hardware or computer-executable software) that can facilitate intelligent hospital scenario simulation and emulation. In various aspects, such computerized tool can comprise a modeling component, a simulation component, an analysis component, or an artificial intelligence component. In various cases, it can be desired to simulate various hospital scenarios using various configurations of hospital resources to identify operational bottlenecks in hospital operations. As described herein, the computerized tool can facilitate such simulation and analysis.

Various embodiments described herein can be employed to use hardware or software to solve problems that are highly technical in nature (e.g., to facilitate intelligent hospital scenario simulation and emulation), that are not abstract and that cannot be performed as a set of mental acts by a human. Further, some of the processes performed can be performed by a specialized computer (e.g., language embedding models, language embedding models) for carrying out defined acts related to hospital scenario simulation and emulation. For example, such defined acts can include: generating, by a system operatively coupled to a processor, a configuration of resources or patient flow of one or more hospital units; simulating, by the system, a scenario in the one or more hospital units using the configuration to generate simulation data; and generating, by the system, action plan recommendations for operational bottlenecks detected in the configuration based on the simulation data. In various aspects, such defined acts can further include determining, by the system, via a large language model (LLM), the configuration based on natural language input or machine-readable input.

Such defined acts are not performed manually by humans. Indeed, neither the human mind nor a human with pen and paper can: electronically execute an artificial intelligence model (e.g., language embedding models, language embedding models) on simulation data, so as to cause the artificial intelligence model to generate recommendations of resource utilization that can improve hospital operations or generate summaries and data visuals that ease or improve a user’s viewing of resulting simulation data; and electronically render a GUI that displays recommendations, summaries, and data visuals. Indeed, an artificial intelligence model (e.g., a language embedding model, a language embedding model) is an inherently-computerized construct that simply cannot be meaningfully executed or trained in any way by the human mind without computers. Similarly, a GUI is an inherently-computerized construct that is electronically rendered or projected onto a computer screen and that cannot be meaningfully implemented in any way by the human mind without computers. Accordingly, a computerized tool that can electronically render a GUI to obtain input from a user and aid a user’s understanding of hospital scenario simulation data based on recommendations, summaries, or data visuals generated via an artificial intelligence model is likewise inherently-computerized and cannot be implemented in any sensible, practical, or reasonable way without computers.

Furthermore, various embodiments described herein can control real-world tangible devices based on the disclosed teachings. For example, various embodiments described herein can electronically train or execute real-world artificial intelligence (AI) models on real-world hospital data, and can electronically render real-world GUIs on real-world computer screens.

It should be appreciated that the herein figures and description provide non-limiting examples of various embodiments and are not necessarily drawn to scale.

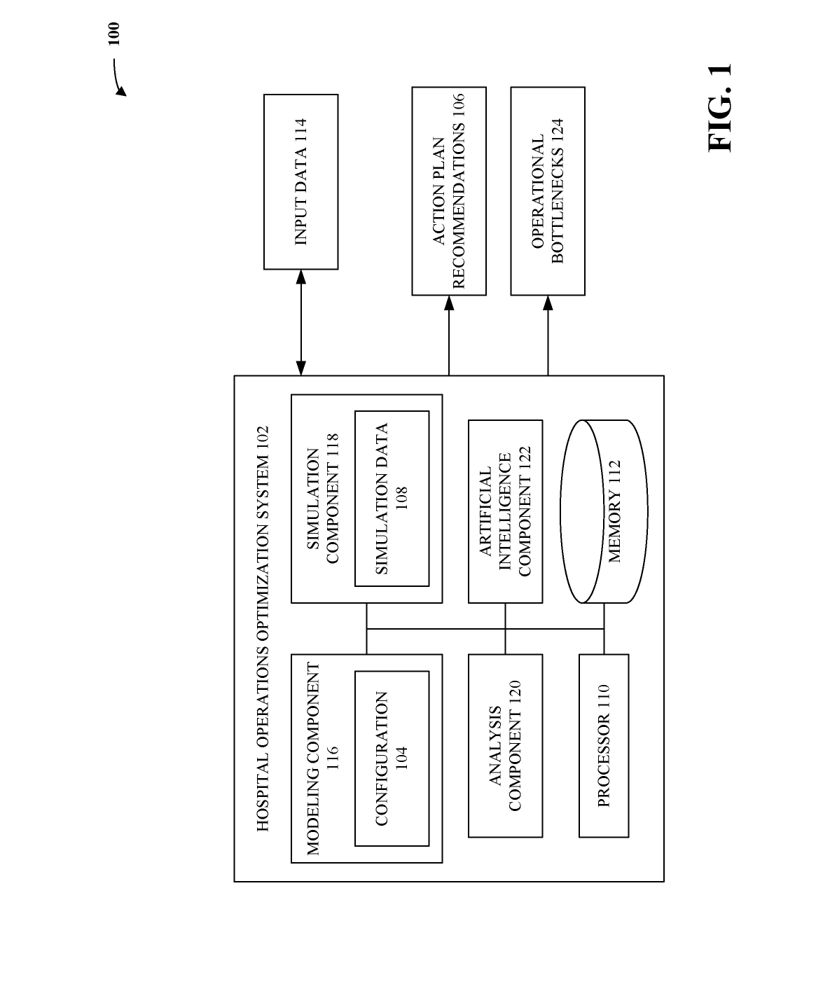

FIG. 1 illustrates a block diagram of an example, non-limiting system 100 that can facilitate intelligent hospital scenario simulation and emulation in accordance with one or more embodiments described herein.

In various embodiments, the hospital operations optimization system 102 can comprise a processor 110 (e.g., computer processing unit, microprocessor) and a non-transitory computer-readable memory 112 that is operably or operatively or communicatively connected or coupled to the processor 110. The non-transitory computer-readable memory 112 can store computer-executable instructions which, upon execution by the processor 110, can cause the processor 110 or other components of the hospital operations optimization system 102 (e.g., modeling component 116, simulation component 118, analysis component 120, artificial intelligence component 122) to perform one or more acts. In various embodiments, the non-transitory computer-readable memory 112 can store computer-executable components (e.g., modeling component 116, simulation component 118, analysis component 120, artificial intelligence component 122), and the processor 110 can execute the computer-executable components.

In various embodiments, the hospital operations optimization system 102 can comprise a modeling component 116. In various aspects, the modeling component 116 can electronically receive or otherwise electronically access input data 114. In various instances, the modeling component 116 can electronically retrieve the input data 114 from any suitable centralized or decentralized data structures (not shown) or from any suitable centralized or decentralized computing devices (not shown), whether local to or remote from the modeling component 116. As a non-limiting example, the modeling component 116 can electronically retrieve the input data 114 from whatever computing devices (e.g., desktop computer, laptop computer, smart phone, tablet) that are responsible for maintaining, storing, or collecting the input data 114. In any case, the modeling component 116 can electronically obtain or access the input data 114 and can generate a configuration 104 based on the input data 114. In various embodiments, the input data 114 can be any suitable electronic data exhibiting any suitable format, size, or dimensionality and indicating, conveying, or otherwise specifying configuration 104.

In various instances, the input data 114 can include natural language input (e.g., human understandable text, commands, or descriptions). In other instances, the input data 114 can include machine-readable input (e.g., structured data formats such as JSON, XML, CSV, API-generated inputs). In still other cases, the input data 114 can include a combination of natural language input and machine-readable input.

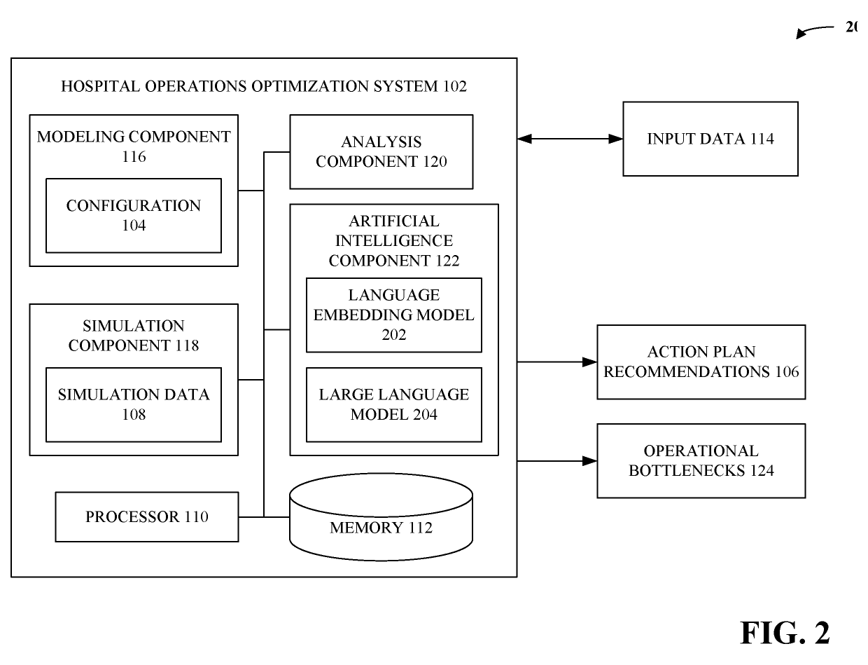

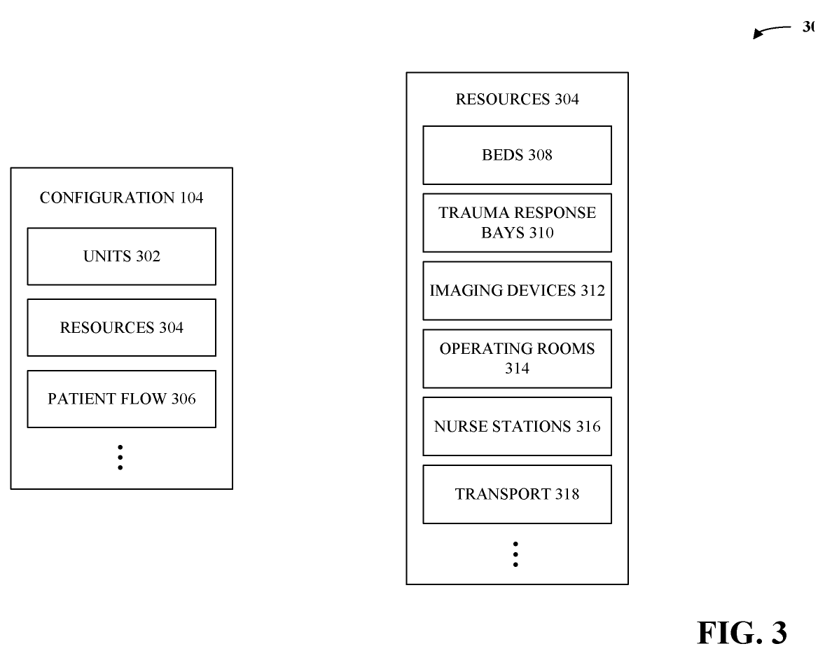

In various embodiments, based on input data 114, modeling component 116 can generate configuration 104. In various aspects, configuration 104 can be associated with a hospital. That is, configuration 104 can describe a combination of resources, departments, units, patient flow, or other operational parameters of the hospital in a particular scenario. In various cases, the modeling component 116 can, as described herein, engage artificial intelligence component 122 to generate the configuration 104 based on the input data 114. Various non-limiting aspects are described with respect to FIGS. 2 and 3.

In various embodiments, the hospital operations optimization system 102 can comprise a simulation component 118. In various aspects, the simulation component 118 can, as described herein, simulate a scenario in the hospital using configuration 104, thereby yielding simulation data 108. In various embodiments, the simulation data 108 can be any suitable electronic data exhibiting any suitable format, size, or dimensionality and indicating, conveying, or otherwise specifying any data that describes the hospital. In various aspects, simulation data 108 can include key operational metrics, resource utilization, patient flow patterns, or other hospital performance metrics. As a non-limiting example, simulation data 108 can include event logs, time-series data representing patient arrivals, distributions of treatment durations, capacity utilization rates of hospital units, transfer frequencies between units, or aggregated measures of system efficiency. In any case, simulation component 118 can simulate the scenario in the hospital using configuration 104 to produce simulation data 108 as output.

In various aspects, simulation component 118 can use any suitable simulation engine to perform the simulation of configuration 104. In various instances, the simulation engine can employ discrete event simulation, agent-based modeling, system dynamics, or any other suitable computational modeling techniques to accurately represent hospital operations specific to the hospital being simulated.

In various embodiments, the hospital operations optimization system 102 can comprise an analysis component 120. In various instances, the analysis component 120 can, as described herein, detect operational bottlenecks 124 based on simulation data 108. For instance, the analysis component 120 can identify resource constraints, such as a shortage of available trauma beds in an emergency department, excessive wait times for MRI scans, or ICU capacity limitations. By evaluating simulation data 108, the analysis component 120 can determine causes of operational bottlenecks 124. In other words, the analysis component 120 can detect operational bottlenecks 124 in simulation data 108. For instance, analysis component 120 can determine that insufficient resources are causing operational bottlenecks 124, such as an inadequate number of nursing stations. These examples are non-limiting, and any hospital resources (e.g., clinicians, nurses, health devices, beds, and non-health devices) where more is required than available can be considered within operational bottlenecks 124. Similarly, analysis component 120 can detect under-used hospital resources, which can indicate an over-investment, and can also be considered within operational bottlenecks 124.

In response to detecting operational bottlenecks 124 based on simulation data 108, the analysis component 120 can generate action plan recommendations 106. Specifically, the action plan recommendations 106 can be any suitable recommendations or suggestions that the hospital can implement to improve hospital operations and improve patient care (e.g., improve operational efficiency, prevent operational bottlenecks, decrease patient wait times, reduce operation costs, improve resource usage, address underused hospital resources). As a non-limiting example, the analysis component 120 can recommend increasing the number of beds in a hospital unit to decrease patient wait times and prevent a bottleneck in patient arrivals. In various aspects, the action plan recommendations 106 can comprise any suitable number of recommendations or suggestions that the hospital can implement to improve hospital operations.

In various aspects, the analysis component 120 can engage the artificial intelligence component 122 to generate action plan recommendations 106 for improving hospital operations. As a non-limiting example, the analysis component 120 can execute an AI model on simulation data 108 to generate the action plan recommendations 106. To help cause the action plan recommendations 106 generated from the simulation data 108 described herein to be accurate, the AI model can first undergo training. In various aspects, the computerized tool described herein can facilitate such training in any suitable fashion (e.g., in supervised fashion or unsupervised fashion) based on any suitable training dataset.

In any instance, the analysis component 120 can generate action plan recommendations 106 based on the scenario simulation of the hospital using configuration 104 (e.g., based on simulation data 108) to recommend or suggest changes in configuration 104 or resource utilization of the hospital that would improve hospital operations and thus improve patient care.

In various embodiments, the analysis component 120 can further apply the action plan recommendations 106 to operational configuration data associated with one or more hospital units. Such operational configuration data can include, for example, non-clinical parameters defining staffing assignments, bed or capacity allocations, patient flow routing rules, scheduling constraints, or other operational settings used to model or manage hospital operations. Applying the action plan recommendations 106 to the operational configuration data can enable updated configurations to be stored, versioned, or made available for subsequent simulation, comparison, or review by hospital operations personnel.

FIG. 2 illustrates a block diagram of an example, non-limiting system 200 including a language embedding model and a large language model (LLM) that facilitates intelligent hospital scenario simulation and emulation in accordance with one or more embodiments described herein. As shown, the system 200 can, in some cases, comprise the same components as the system 100, and can further comprise a language embedding model 202 and an LLM 204.

In various embodiments, the artificial intelligence component 122 can electronically store, maintain, control, or otherwise access the language embedding model 202. In various aspects, the language embedding model 202 can exhibit any suitable internal architecture. For instance, the language embedding model 202 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers. In addition to the neural network described above, the language embedding model 202 can comprise one or more pre-processing and post-processing layers that can segment input data into portions and can process non-English or non-natural language content, including numerical data and structured simulation configuration data (e.g., in JSON or YAML). These one or more pre-processing and post-processing layers can apply specialized handling to whitespace characters and numeric values (e.g., in a manner distinct from conventional natural language processing of English text) and transform the input data into a representation compatible with processing by the neural network of the language embedding model 202. No matter the internal architecture of the language embedding model 202, the language embedding model 202 can be configured to generate numerical representations (vector embeddings) based on input data. Accordingly, artificial intelligence component 122 can electronically execute the language embedding model 202 on the input data 114, thereby yielding vector embeddings that represent input data 114. In various aspects, language embedding model 202 can be trained using any suitable training paradigm (e.g., supervised learning, unsupervised learning). In various cases, language embedding model 202 can be pre-trained.

Likewise, the artificial intelligence component 122 can electronically store, maintain, control, or otherwise access the LLM 204. In various aspects, the LLM 204 can exhibit any suitable internal architecture. For instance, the LLM 204 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

No matter the internal architecture of the LLM 204, the LLM 204 can be configured to generate a configuration representing a hospital scenario based on inputted vector embeddings. Accordingly, artificial intelligence component 122 can electronically execute the language embedding model 202 on the vector embeddings representing input data 114, thereby yielding configuration 104. In various aspects, LLM 204 can be trained using any suitable training paradigm (e.g., supervised learning, unsupervised learning). In various cases, language embedding model 202 can be pre-trained.

In various embodiments, the artificial intelligence component 122 can execute the language embedding model 202 on input data 114, thereby producing vector embeddings of input data 114. Thus, artificial intelligence component 122 can execute the LLM 204 on the vector embeddings to produce configuration 104 as output.

In some cases, the artificial intelligence component 122 can directly execute the LLM 204 on input data 114 to generate configuration 104. For instance, if the input data 114 consists of machine-readable input, the artificial intelligence component 122 can refrain from executing language embedding model 202 on input data 114.

In various aspects, the language embedding model 202 can reduce the number of data dimensions of input data 114 to improve computational efficiency without losing key data features (e.g., retaining relationships between words or phrases to ensure that semantic and contextual features of the data are not lost). In this way, LLM 204 can understand and interpret the input data 114 accurately to determine configuration 104 according to a user’s desired settings.

As a non-limiting example, the input data 114 can be natural language user input, such as “Configure the trauma unit to have 5 treatment beds and 3 stabilization bays”. The artificial intelligence component 122 can execute the language embedding model 202 on the natural language user input, thereby yielding compact vector embeddings. Accordingly, the artificial intelligence component 122 can execute the LLM 204 on the vector embeddings to generate configuration 104, such that configuration 104 indicates 5 treatment beds and 3 stabilization bays in the trauma unit. Thus, simulation component 118 can simulate a scenario in the trauma unit where there are 5 treatment beds and 3 stabilization bays. Thereafter, based on simulation data 108 resulting from such simulation, the analysis component 120 can identify if comprising 5 treatment beds and 3 stabilization bays results in operational bottlenecks 124 in the trauma unit (e.g., there are not enough stabilization bays, causing delays in patient care).

FIG. 3 illustrates an example, non-limiting block diagram showing a configuration and a plurality of resources of a hospital in accordance with one or more embodiments described herein.

In various aspects, as shown, the configuration 104 can comprise units 302. In various embodiments, units 302 can be one or more scalars, one or more vectors, one or more matrices, one or more tensors, one or more character strings, or any suitable combination thereof that can indicate, convey, or otherwise represent one or more units in the hospital that are to be simulated. As a non-limiting example, units 302 can specify the emergency department in a hospital. As another non-limiting example, units 302 can specify an ICU and a cardiac care unit in a hospital. As yet another non-limiting example, units 302 can include but are not limited to any of the following: emergency department or response unit (ED or ER), triage unit, trauma unit, pediatric unit, ICU, intermediate care unit (IMC), ambulatory surgery unit (ASU), general admission (GA), medical surgery unit (Med-Surg), labor and delivery unit (L&D), mother-baby unit or newborn nursery (MBU), neonatal intensive care unit (NICU), cardiology unit, neurology unit, or oncology unit. Units 302 can further include subunits of the aforementioned units. For example, units 302 can include subunits of the ICU such as a burn unit, cardiac intensive care unit (CICU), or cardiothoracic intensive care unit (CTICU).

In various embodiments, the configuration 104 can further comprise patient flow 306. In various aspects, patient flow 306 can be one or more scalars, one or more vectors, one or more matrices, one or more tensors, one or more character strings, or any suitable combination thereof that can indicate, convey, or otherwise represent patient flow or traffic patterns of patients in the hospital. As a non-limiting example, patient flow 306 can indicate an average number of patients that visit each of the units 302 over a defined period of time. As another non-limiting example, patient flow 306 can indicate patient arrival rates. As yet another non-limiting example, patient flow 306 can indicate transition rates of patients within a unit of the units 302.

In various instances, the modeling component 116 can model the patient flow 306 based on user input (e.g., user input received via a GUI) or input data 114. As a non-limiting example, a user can input or select a statistical model to model the patient flow 306. In other instances, the modeling component 116 can model the patient flow 306 based on historical electronic medical records (EMR). As a non-limiting example, the modeling component 116 can model the patient flow 306 based on past admission rates, treatment durations, or discharge patterns.

In some cases, the modeling component 116 can model the patient flow 306 based on live EMR feeds. As a non-limiting example, the modeling component 116 can model the patient flow 306 based on real-time patient intake, bed occupancy, or treatment progress data. In various aspects, the modeling component 116 can continuously access the live EMR feeds to dynamically adjust patient flow predictions and adjust thus adjust patient flow 306 accordingly. This can enable emulation of the hospital for detecting operational bottlenecks 124 during hospital operations. Thus, the action plan recommendations 106 determined based on operational bottlenecks 124 can be implemented or applied during hospital operations to prevent the operational bottlenecks 124 from occurring.

In still other cases, the modeling component 116 can model the patient flow 306 based on AI-generated patient flows. Particularly, the modeling component 116 can engage the artificial intelligence component 122 to generate patient flows. As a non-limiting example, the artificial intelligence component 122 can simulate patient movement scenarios under varying conditions, such as seasonal fluctuations, emergency surges, or staffing changes. In some cases, the artificial intelligence component 122 can generate the AI-generated patient flows based on the historical EMR or live EMR feeds.

In various embodiments, the configuration 104 can further comprise resources 304. In various aspects, resources 304 can be one or more scalars, one or more vectors, one or more matrices, one or more tensors, one or more character strings, or any suitable combination thereof that can indicate, convey, or otherwise represent resources of the units 302 of the hospital that are to be simulated.

In various embodiments, resources 304 can include beds 308 in units 302. In various instances, the beds 308 can be one or more scalars, one or more vectors, one or more matrices, one or more tensors, one or more character strings, or any suitable combination thereof that can indicate, convey, or otherwise represent a number of beds that are in one or more of units 302. As a non-limiting example, beds 308 can indicate a total number of beds in units 302. As another non-limiting example, beds 308 can indicate a number of beds in the ICU of the hospital and a number of beds in the ER.

In various embodiments, resources 304 can include trauma response bays 310 of units 302. In various instances, the trauma response bays 310 can be one or more scalars, one or more vectors, one or more matrices, one or more tensors, one or more character strings, or any suitable combination thereof that can indicate, convey, or otherwise represent a number of trauma response bays that are in one or more of units 302.

In various embodiments, resources 304 can include imaging devices 312 of units 302. In various instances, the imaging devices 312 can be one or more scalars, one or more vectors, one or more matrices, one or more tensors, one or more character strings, or any suitable combination thereof that can indicate, convey, or otherwise represent a number and type of imaging devices that are in one or more of units 302. As a non-limiting example, imaging devices 312 can indicate that there are three computed tomography (CT) scanners and four MRI machines in units 302. As another non-limiting example, imaging devices 312 can indicate a number of each type of imaging device in units 302 (e.g., two X-ray machines in the non-trauma unit, three CT scanners in the trauma unit). In various cases, the imaging devices 312 can include but are not limited to the following imaging devices: CT, MRI, X-ray, ultrasound (ULS).

In various embodiments, resources 304 can include operating rooms 314 of units 302. In various instances, the operating rooms 314 can be one or more scalars, one or more vectors, one or more matrices, one or more tensors, one or more character strings, or any suitable combination thereof that can indicate, convey, or otherwise represent a number of operating rooms that are in one or more of units 302.

In various embodiments, resources 304 can include nurse stations 316 of units 302. In various instances, the nurse stations 316 can be one or more scalars, one or more vectors, one or more matrices, one or more tensors, one or more character strings, or any suitable combination thereof that can indicate, convey, or otherwise represent a number of nurse stations that are in one or more of units 302.

In various embodiments, resources 304 can include transports 318 of units 302. In various instances, the transports 318 can be one or more scalars, one or more vectors, one or more matrices, one or more tensors, one or more character strings, or any suitable combination thereof that can indicate, convey, or otherwise represent a number of transports that are in one or more of units 302. As a non-limiting example, the transports 318 can include but are not limited to ambulances, helicopters, or wheelchairs.

Note that these are non-limiting examples of configuration 104, and that configuration 104 can include any suitable operational parameters associated with the hospital. That is, configuration 104 is not limited to units 302, resources 304, and patient flow 306. For instance, configuration 104 can comprise any suitable type of operational parameter associated with the hospital. As non-limiting examples, configuration 104 can further include departments (e.g., number and type of departments), protocols (e.g., clinical rules, care pathways, policies, compliance requirements), schedules (e.g., staff hours), hospital layout (e.g., resource distribution among units, physical hospital layout of rooms) or time (e.g., season, time of day, day of the week).

Further note that these are non-limiting examples of resources 304, and that resources 304 can include any suitable equipment, resources, personnel, etc. to be simulated, such as bedside monitoring devices, defibrillators, staffing etc. That is, resources 304 is not limited to beds 308, trauma response bays 310, imaging devices 312, operating rooms 314, nurse stations 316, and transports 318. For instance, resources 304 can comprise any suitable type of resource associated with the hospital. As non-limiting examples, resources 304 can further include staffing (e.g., number of clinicians, nurses, or technicians), pharmaceuticals (e.g., type and number of medications), ventilators (e.g., number of ventilators), or surgical instruments (e.g., type and number of surgical instruments).In various instances, resources 304 can include any suitable number of resource types (e.g., 3 resources, 8 resources, 15 resources, 20 resources).

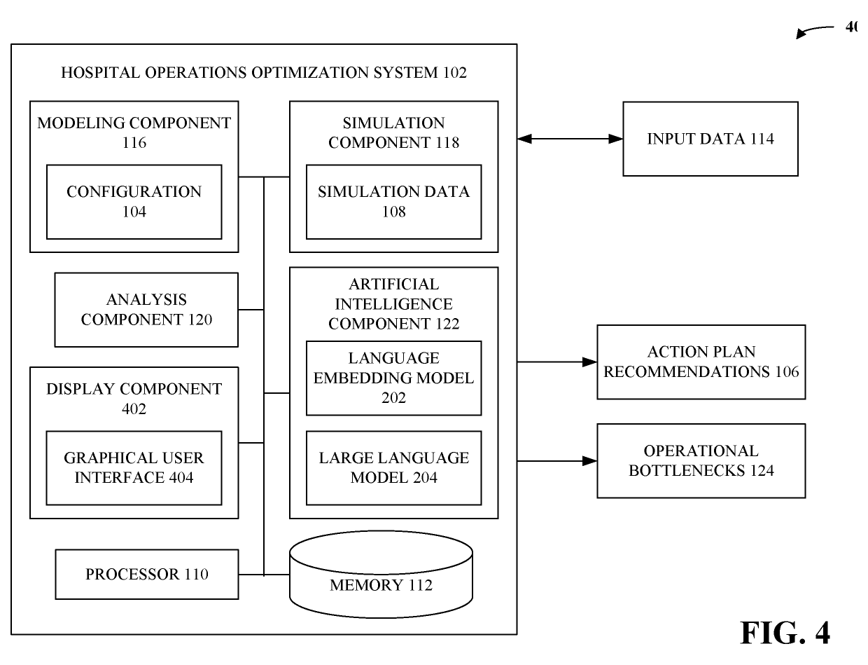

FIG. 4 illustrates a block diagram of an example, non-limiting system 400 including a display component and a graphical user interface (GUI) that facilitates intelligent hospital scenario simulation and emulation in accordance with one or more embodiments described herein. As shown, the system 400 can, in some cases, comprise the same components as the system 200, and can further comprise a display component 402 and a graphical user interface 404 (hereafter “GUI 404”).

In various embodiments, the hospital operations optimization system 102 can further comprise display component 402. In various instances, the display component 402 can, as described herein, visually render GUI 404.

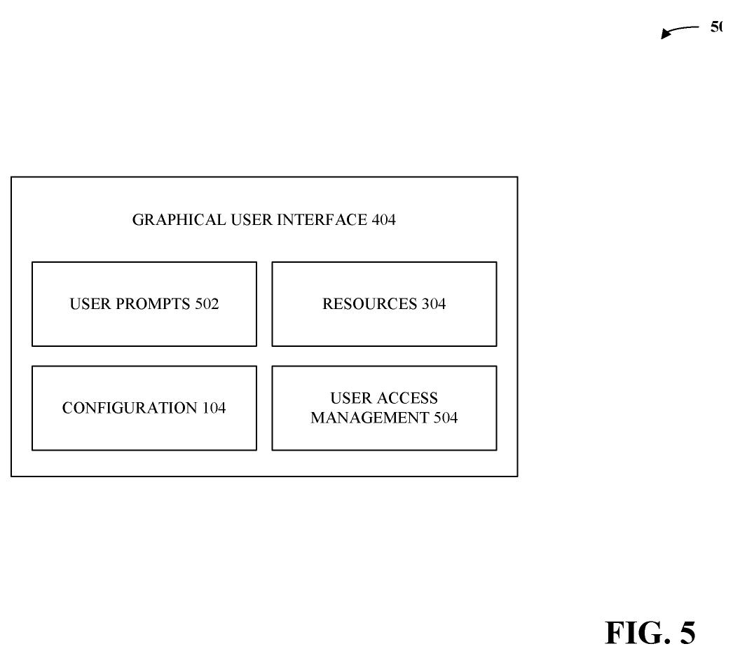

In various embodiments, the display component 402 can electronically generate the GUI 404. In various aspects, the display component 402 can visually render, or otherwise cause to be visually rendered, the GUI 404 on any suitable electronic display of any suitable computing device. As a non-limiting example, the display component 402 can cause the GUI 404 to be rendered on an electronic computer screen of any suitable smart phone device (e.g., a smart phone of the medical patient). As another non-limiting example, the display component 402 can cause the GUI 404 to be rendered on an electronic computer screen of any suitable wearable device (e.g., smart watch of the medical patient, smart glasses of the medical patient). As even another non-limiting example, the display component 402 can cause the GUI 404 to be rendered on an electronic computer screen of any suitable hospital console device (e.g., a bedside hospital monitor that is near the medical patient, a staff monitor). Various non-limiting aspects are described with respect to FIGS. 5, 8, and 12-15.

It is to be appreciated that any other suitable aspects or details associated with clinical simulation GUIs can be implemented in conjunction with the GUI 404. As some non-limiting examples, the GUI 404 can implement: any suitable data packaging, analysis, or exporting techniques (e.g., structured data exports in formats such as CSV, JSON, or XML, integration with third-party analytics platforms, real-time visualization of simulation data 108); or any suitable augmented reality or virtual reality techniques (e.g., if scanned images of the hospital are available, they can be leveraged to construct a two-dimensional or three-dimensional virtual model of the hospital).

FIG. 5 illustrates an example, non-limiting block diagram 500 showing the GUI 404 that facilitates intelligent hospital scenario simulation and emulation in accordance with one or more embodiments described herein.

In various embodiments, the modeling component 116 can detect gaps in configuration 104. That is, based on input data 114, there can be insufficient information to determine configuration 104. For instance, a user can enter input data 114, however, input data 114 may not indicate or include information about the patient flow 306. In various aspects, the modeling component 116 can detect that there is insufficient information to define patient flow 306 for simulation. In any case, in response to detecting gaps in configuration 104, the modeling component 116 can generate, via LLM 204, user prompts 502.

In various instances, the modeling component 116 can generate, via LLM 204, any suitable number of user prompts 502 to prompt a user to provide sufficient information to generate configuration 104. In other words, the user prompts 502 can facilitate retrieval of additional information from a user to fill in the gaps in configuration 104. In various aspects, the user prompts can include, but are not limited to, questions, suggestions, or commands. For instance, the user prompts 502 can be simple questions that can enable non-technical subject matter experts (e.g., care nurses, imaging technicians) to help define the configuration 104. As a non-limiting example, the user prompts 502 can be simple questions such as “How long does it take to perform this step?” to assist in determining patient flow 306. As another non-limiting example, the user prompts 502 can include a number of choices from which a user can select (e.g., select between low patient flow, medium patient flow, or high patient flow).

Note that these are mere non-limiting examples and that the GUI 404 can display any suitable questions, prompts, or suggestions that can facilitate complete generation of configuration 104.

In any case, the modeling component 116 can generate, via LLM 204, the user prompts 502, and the display component 402 can cause the GUI 404 to electronically depict or illustrate the user prompts 502. Therefore, a user can provide, via the GUI 404, additional input that the modeling component 116 can utilize to generate configuration 104.

In various aspects, the display component 402 can cause the GUI 404 to electronically depict or illustrate resources 304. Therefore, a user can alter, change, view, or otherwise interact with, via the GUI 404, resources 304 so as to enable a user to create any desired configuration 104. That is, further to providing additional information to input data 114 via user prompts 502, the user can also manually make any desired changes to resources 304 (e.g., to reflect the current resources of a hospital, to reflect the resources of a hospital at a past time, to reflect the resources of a hospital in an imaginary scenario). As a non-limiting example, the user can view or change, via GUI 404, the number or type of hospital units. As another non-limiting example, the user can view or change, via GUI 404, the number of resources 304 (e.g., the number of beds 308). As still another non-limiting example, the user can view or change, via GUI 404, patient admission or discharge rates.

In any case, the display component 402 can cause the GUI 404 to electronically depict or illustrate configuration 104 generated based on input data 114, resources 304 (e.g., changes to resources 304 from user input), and additional information received based on the user prompts 502.

In various embodiments, the user can control, access, or otherwise view, via the GUI 404, user access management 504. In various aspects, the user (e.g., an administrator) can view or change access settings for various users via user access management 504. As a non-limiting example, an administrator can restrict or grant, via the GUI 404, access to employees in user access management 504. In other instances, the user can request changes to access settings via user access management 504. As a non-limiting example, the user can request, via GUI 404, access to particular resources or information.

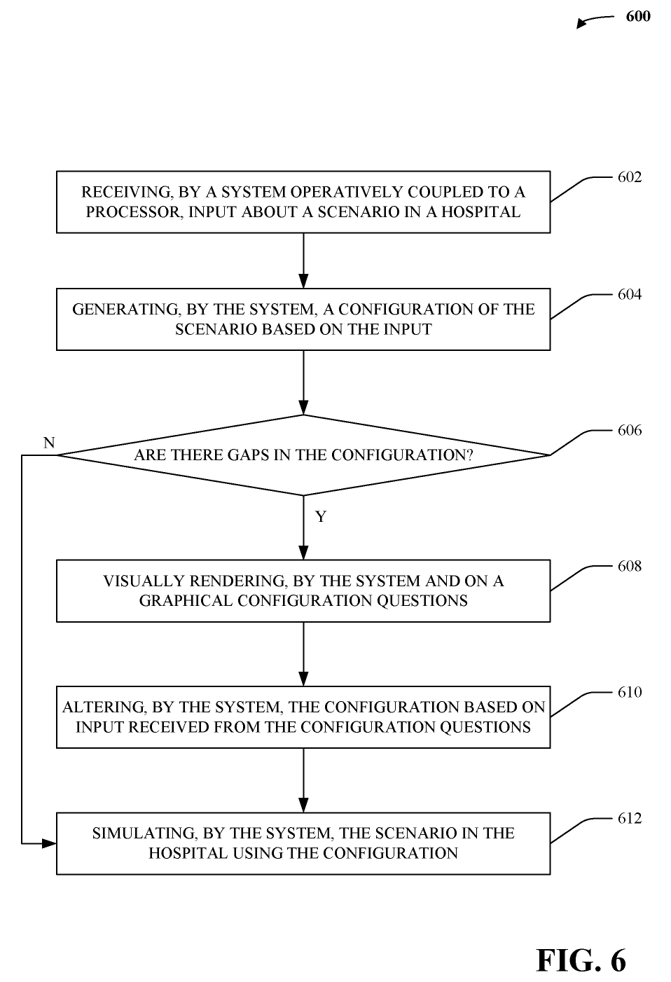

FIG. 6 illustrates a flow diagram of an example, non-limiting computer-implemented method 600 that can facilitate intelligent hospital scenario simulation and emulation in accordance with one or more embodiments described herein. In various cases, the hospital operations optimization system 102 can facilitate the computer-implemented method 600.

In various embodiments, act 602 can include receiving, by a system (e.g., via modeling component 116) operatively coupled to a processor (e.g., 110), input about a scenario in a hospital.

In various aspects, act 604 can include generating, by the system (e.g., via modeling component 116), a configuration of the scenario based on the input.

In various instances, act 606 can include determining if there are gaps in the configuration. If yes (e.g., there are gaps in the configuration), the computer-implemented method 600 can proceed to act 608. If no (e.g., there are no gaps in the configuration), the computer-implemented method 600 can proceed to act 612.

In various instances, act 608 can include visually rendering, by the system (e.g., via display component 402) and on a graphical user interface (e.g., 404), configuration questions (e.g., 404).

In various aspects, act 610 can include altering, by the system (e.g., via modeling component 116), the configuration based on input received from the configuration questions.

In various aspects, act 612 can include simulating, by the system (e.g., via simulation component 118), the scenario in the hospital using the configuration.

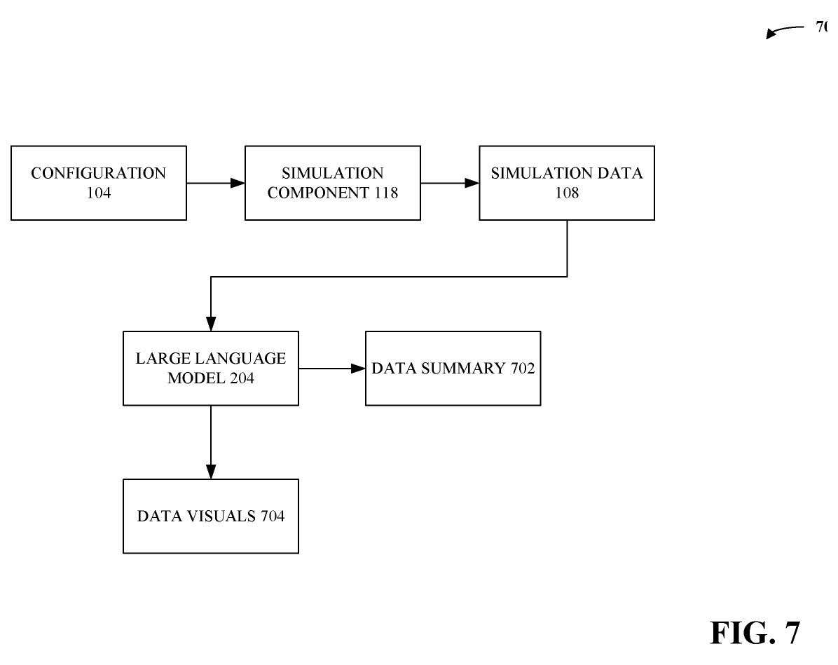

FIG. 7. illustrates an example, non-limiting block diagram showing generation of simulation summaries and data visuals in accordance with one or more embodiments described herein.

In various embodiments, the simulation component 118 can electronically access configuration 104. Thereafter, the simulation component 118 can simulate a scenario in a hospital using configuration 104, thereby producing simulation data 108.

In many cases, the simulation data 108 is typically not easily understandable to users, particularly non-technical stakeholders such as clinicians, hospital administrators, or operational staff. The simulation data 108 data can be highly granular, consisting of raw numerical outputs, statistical probabilities, or complex system dynamics that require interpretation. To enhance usability, the analysis component 120 can execute the LLM 204 on the simulation data 108 to produce a data summary 702 and/or data visuals 704. Specifically, the analysis component 120 can generate data summary 702 and data visuals 704 using natural language to improve understandability to users, thus enabling more efficient decision-making regarding improving hospital operations.

For instance, data summary 702 can include natural language describing operational trends, such as average patient wait times, unit utilization rates, or bottleneck locations. As another example, data visuals 704 can include charts, graphs, or flow diagrams that illustrate patient flow patterns, capacity constraints, or comparative outcomes across alternate scenarios or configurations.

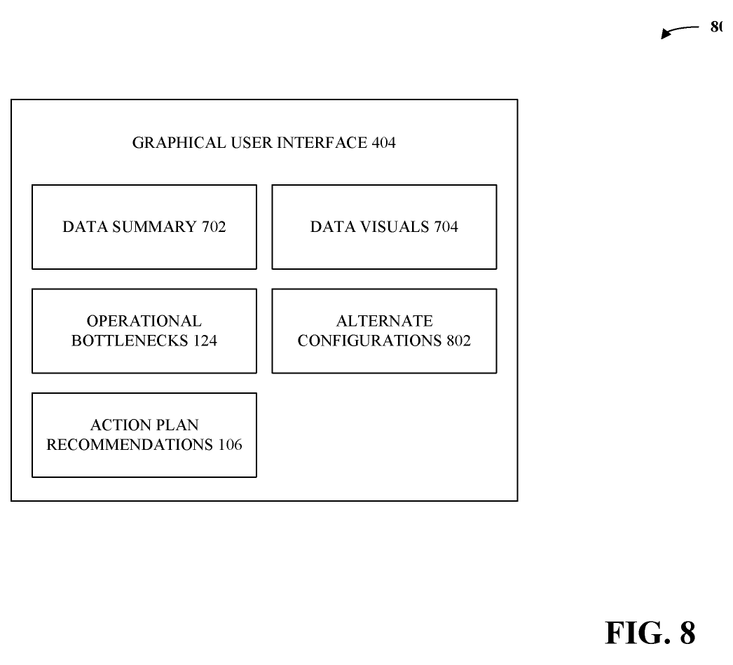

FIG. 8 illustrates an example, non-limiting block diagram showing GUI 404 that facilitates intelligent hospital scenario simulation and emulation in accordance with one or more embodiments described herein.

In various aspects, the display component 402 can cause the GUI 404 to electronically depict or illustrate data summary 702 and data visuals 704. Additionally, in response to the analysis component 120 detecting operational bottlenecks 124 from the simulation data 108, the display component 402 can cause the GUI 404 to electronically depict or illustrate the operational bottlenecks 124. Particularly, the analysis component 120 can generate, via LLM 204, textual or visual descriptions that indicate, represent, or convey the operational bottlenecks 124.

In various embodiments, based on simulation data 108, the analysis component 120 can generate alternate configurations 802. The alternate configuration 802 can comprise different configurations of the hospital scenario that can potentially resolve or address the operational bottlenecks 124. In various aspects, the analysis component 120 can recommend the alternate configurations 802, where the display component 402 can cause the GUI 404 to electronically depict or illustrate alternate configuration 802. As a non-limiting example, the analysis component 120 can recommend the following alternate configuration: “Since trauma bays will be full, consider moving 2 surgeons and 4 nurses from the non-trauma unit to the trauma unit”. As another non-limiting example, the analysis component 120 can recommend the following alternate configuration: “The neuro-MRI unit is always full for 60% of the time and patient wait time is >40 minutes. Consider putting 2 more MRI machines there”.

In various embodiments, a user can select, via the GUI 404, one or more of the alternate configurations 802. Accordingly, the simulation component 118 can automatically apply the alternate configurations 802 in simulation setup. Thereafter, the simulation component 118 can re-simulate the scenario with the alternate configurations 802. Thus, the analysis component 120 can generate and provide analysis that describes or indicates the differences in simulation data 108 between the alternate configurations 802. As a non-limiting example, in addition to data summary 702 and data visuals 704, the GUI 404 can display the following analysis: “The ICU unit with the newly added 100 beds has 10+ beds free all the time. Consider rerunning the simulation with 90 beds”. In any case, the display component 402 can cause the GUI 404 to electronically depict or illustrate the action plan recommendations 106.

In this way, the analysis component 120 can assess the impact of the action plan recommendations 106 for solving or addressing the operational bottlenecks 124, such as adding more MRI machines, expanding ICU capacity, or optimizing scheduling strategies, to ensure that changes (e.g., investments) in additional resources yield maximum or otherwise improved operational efficiency.

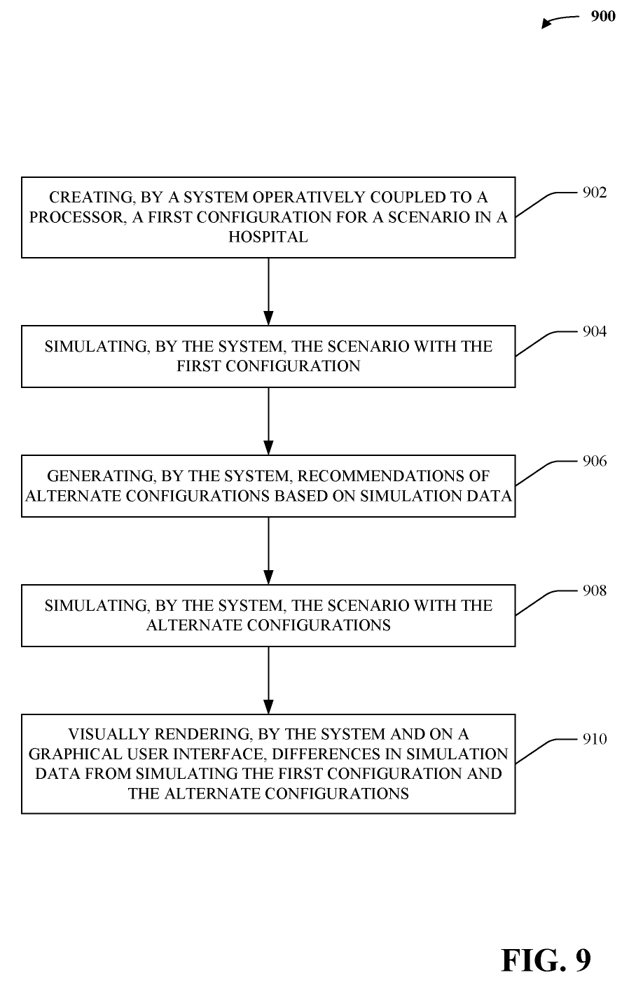

FIG. 9 illustrates a flow diagram of an example, non-limiting computer-implemented method 900 that can facilitate intelligent hospital scenario simulation and emulation in accordance with one or more embodiments described herein. In various cases, the hospital operations optimization system 102 can facilitate the computer-implemented method 900.

In various embodiments, act 902 can include generating, by a system (e.g., via modeling component 116) operatively coupled to a processor (e.g., 110), a first configuration for a scenario in a hospital.

In various aspects, act 904 can include simulating, by the system (e.g., via simulation component 118), the scenario with the first configuration.

In various embodiments, act 906 can include generating, by the system (e.g., via modeling component 116), recommendations of alternate configurations (e.g., 902) based on simulation data (e.g., 404).

In various aspects, act 908 can include simulating, by the system (e.g., via simulation component 118), the scenario with the alternate configurations.

In various instances, act 910 can include visually rendering, by the system (e.g., via display component 402) and on a graphical user interface (e.g., 404), differences in simulation data from simulating the first configuration and the alternate configurations.

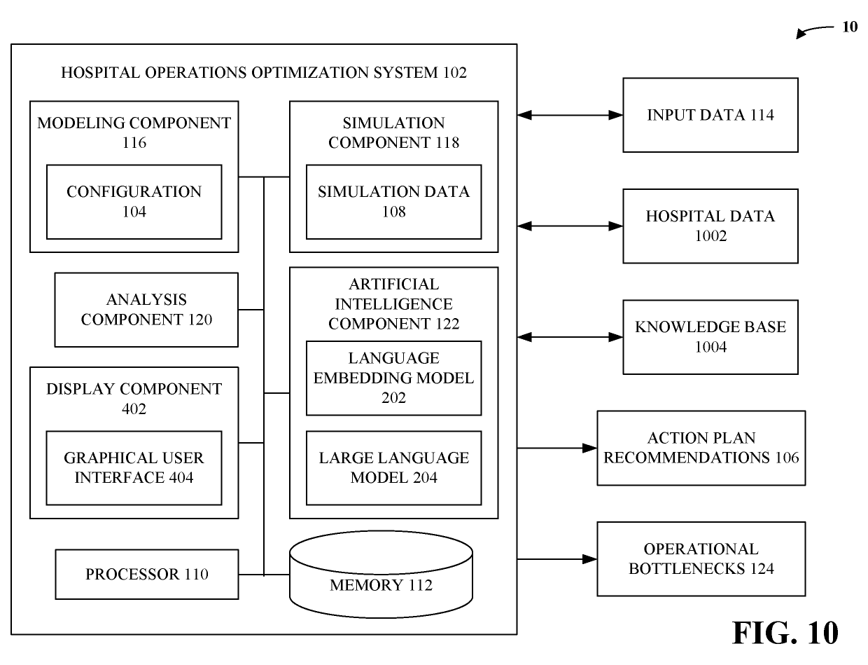

FIG. 10 illustrates a block diagram of an example, non-limiting system 1000 including hospital data and a knowledge base that facilitates intelligent hospital scenario simulation and emulation in accordance with one or more embodiments described herein. As shown, the system 1000 can, in some cases, comprise the same components as the system 400, and can further comprise hospital data 1002 and knowledge base 1004.

As shown, hospital operations optimization system 102 can be electronically integrated, via any suitable wired or wireless electronic connections, with hospital data 1002 and knowledge base 1004. That is, the modeling component 116 can electronically receive or otherwise electronically access the hospital data 1002 and knowledge base 1004.

In various embodiments, the simulation component 118 can simulate a scenario in a hospital based on hospital data 1002 to enhance accuracy and adaptability of the simulation. That is, modeling component 116 can create or change configuration 104 (e.g., units 302, resources 304, patient flow 306) based on hospital data 1002 in addition to input data 114.

In various embodiments, the hospital data 1002 can correspond to or be associated with any suitable hospital (e.g., any suitable healthcare facility). In various aspects, the hospital data 1002 can be one or more scalars, one or more vectors, one or more matrices, one or more tensors, one or more character strings, or any suitable combination thereof that indicates, conveys, or otherwise represents any suitable hospital operations data corresponding to the hospital. As non-limiting examples, the hospital data 1002 can indicate, convey, or otherwise represent patient admission records, discharge summaries, bed occupancy rates, staffing schedules, medical inventory levels, diagnostic imaging reports, real-time patient monitoring data, EMR, treatment workflows, departmental efficiency metrics, or emergency department wait times. In various aspects, the hospital data 1002 can include historical data and current data associated with the hospital.

In various embodiments, following simulation of the scenario (using configuration 104), the simulation component 118 can store simulation data 108 resulting from such simulation in knowledge base 1004 for subsequent access or retrieval. Thus, the analysis component 120 can electronically access the knowledge base 1004 to enable more relevant and accurate action plan recommendations 106 with reduced computational costs.

As a non-limiting example, if a scenario is simulated with a particular configuration, the simulation component 118 can store simulation data 108 corresponding to that simulation in knowledge base 1004. That is, the simulation component 118 can store operational bottlenecks 124, data summary 702, data visuals 704, or action plan recommendations 106 that were generated based on the simulation data 108 in knowledge base 1004. Thus, if the scenario is simulated again using the same configuration (or a similar configuration), simulation component 118 can refrain from simulating the scenario, and the analysis component 120 can instead retrieve the operational bottlenecks 124, data summary 702, data visuals 704, or action plan recommendations 106 from the knowledge base 1004 to reduce computation overhead. As another non-limiting example, analysis component 120 can electronically access the knowledge base 1004 to retrieve simulation data 108 from simulations of a scenario corresponding to alternate configurations 802. Thus, the analysis component 120 can, with greater efficiency, compare simulation data 108 from alternate configurations 802 to determine differences in hospital operations from the alternate configurations 802.

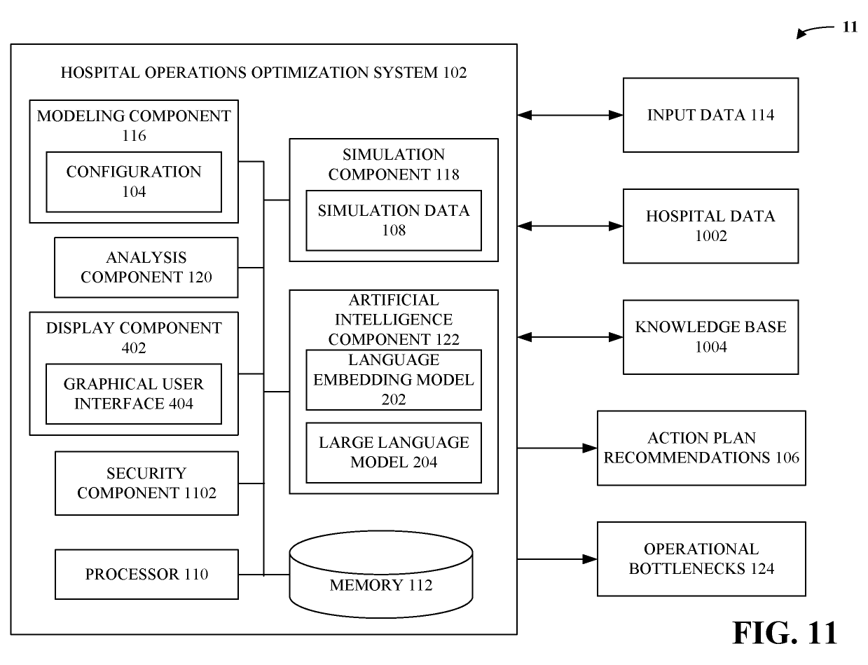

FIG. 11 illustrates a block diagram of an example, non-limiting system 1100 including a security component that facilitates intelligent hospital scenario simulation and emulation in accordance with one or more embodiments described herein. As shown, the system 1100 can, in some cases, comprise the same components as the system 1000, and can further comprise a security component 1102.

In various embodiments, the security component 1102 can employ various security controls or mechanisms that protect end-users (e.g., an administrator or staff of a hospital) and their user data (e.g., hospital data 1002, knowledge base 1004). In various aspects, the security component 1102 can create users (that are associated with one or more hospitals) and manage access permissions to the users. In various aspects, the security controls or mechanisms can include authentication and authorization management of the users to determine who can access what data. Furthermore, the security controls or mechanisms can include segregated data access controls to ensure user data can only be accessed when needed. Moreover, the security controls or mechanisms can include data encryption schemes. Specifically, the security component 1102 can encrypt user data to ensure that the user data is encrypted at rest and in transit. In various aspects, the security controls or mechanisms can further include traffic shaping controls to protect and enable recovery from cyberattacks.

In various embodiments, the security component 1102 can sequence user requests. Specifically, the security component 1102 can sequence user requests via request orchestrators to balance user requests against system capacity and thus enable user experience to be frictionless while protecting against system overload (e.g., excessive demand that could degrade performance).

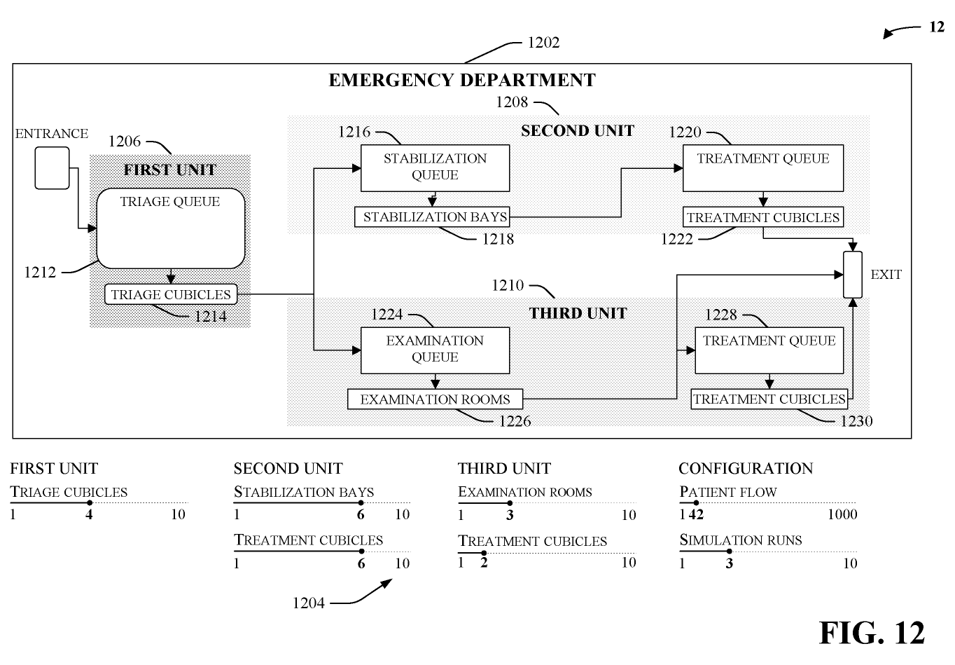

FIG. 12 illustrates a diagram of an example, non-limiting GUI 1200 showing simulation of a scenario using a configuration in accordance with one or more embodiments described herein.

As a non-limiting example, a user may wish to simulate the resources and patient flow of an emergency department 1202 to identify if operational bottlenecks 124 exist. FIG. 12 depicts example, non-limiting GUI 1200 illustrating how a scenario can be visualized for configuration and simulation. As shown in FIG. 12, the emergency department 1202 can comprise a first unit 1206, from which patients can move to a second unit 1208 or a third unit 1210. Patients in the second unit 1208 can move from a stabilization queue 1216 to stabilization bays 1218, to a treatment queue 1220, to treatment cubicles 1222, and then can be discharged. Patients in the third unit 1210 can move from an examination queue 1224, to examination rooms 1226, to a treatment queue 1228, to treatment cubicles 1230, and then can be discharged.

It can be desirable to determine if operational bottlenecks 124 exist to optimize hospital operations in the emergency department 1202 and improve patient care. Accordingly, a user can input, via GUI 404, configuration 1204 for simulating a scenario in the emergency department 1202. In some cases, a user can provide natural language input, from which modeling component 116 can generate, via LLM 204, the configuration 1204. In other cases, the user can select, via GUI 404, settings of configuration 1204. In other instances, the user can modify, via GUI 404, the configuration 1204 after the configuration 1204 is generated based on the user’s natural language input. In any case, simulation component 118 can simulate the scenario in the emergency department 1202 using configuration 1204.

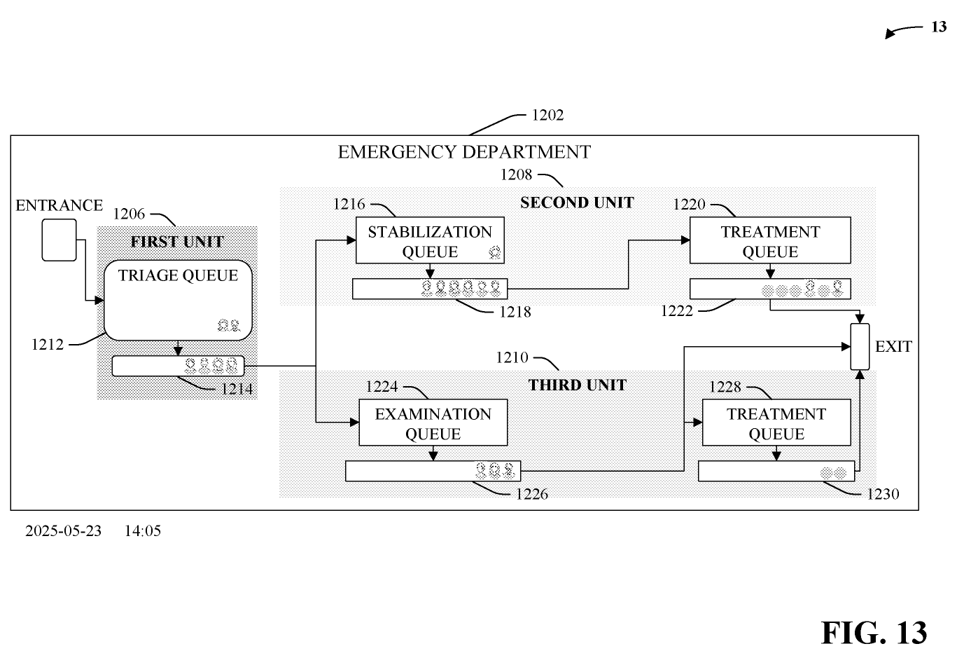

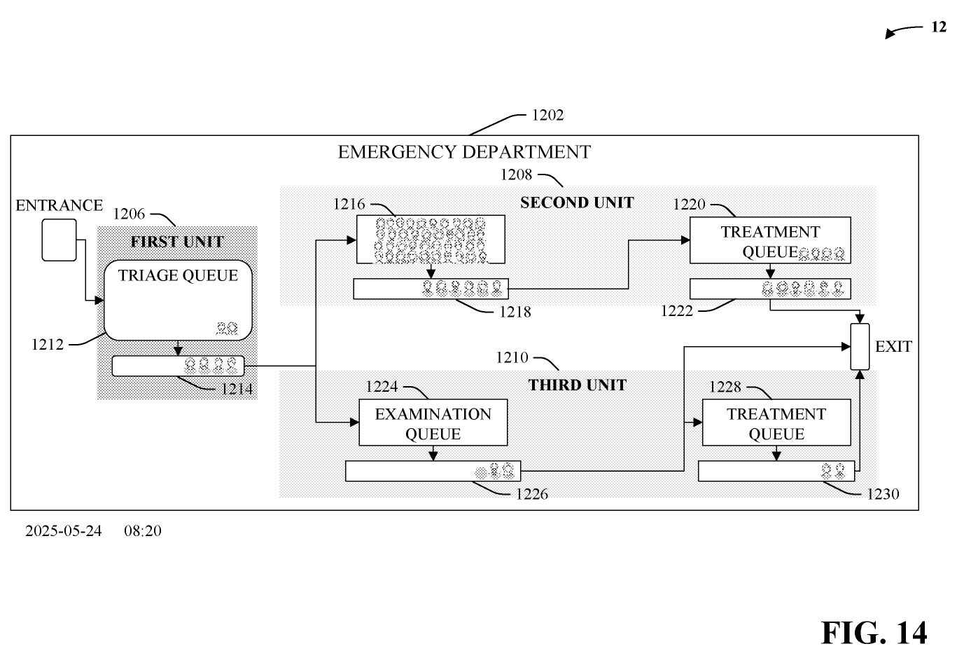

FIGS. 13 and 14 illustrate a diagram of an example, non-limiting GUI 1300 showing a simulation of a scenario in accordance with one or more embodiments described herein. In particular, FIG. 13 illustrates a first timestamp of a simulation of the emergency department 1202, and FIG. 14. illustrates a second timestamp of the emergency department 1202. In other words, FIGS. 13 and 14 depict still frames of a play though of the simulation of a hospital.

As shown in the first timestamp (e.g., 2025-05-23 14:05), the triage cubicles 1214, stabilization bays 1218, and examination rooms 1226 are full, with treatment cubicles 1222 in the second unit 1208 and treatment cubicles 1230 in the third unit 1210 being empty or nearly empty. Furthermore, in the first timestamp, 2 patients are in the triage queue 1212, 1 patient is in the stabilization queue 1216, and 16 patients are in the examination queue 1224. As simulation component 118 simulates the emergency department 1202, the number of patients in each unit can change based on hospital data 1002. As a non-limiting example, based on historical or current patient transfer distributions and arrival rates, simulation component 118 can play out the scenario in the emergency department 1202. For instance, simulation of the scenario from the first timestamp can lead to the second timestamp.

As shown in the second timestamp which is approximately 1 day after the first timestamp (e.g., 2025-05-24 8:20), the stabilization queue 1216 significantly increases to 40 patients, with the treatment queue 1220 and treatment cubicles 1222 in the second unit 1208 becoming full. Conversely, the third unit 1210 has only 2 patients in treatment queue 1228. In such an instance, based on the simulation, the analysis component 120 can, for example, infer or conclude from simulation data 108 that there exist operational bottlenecks 124 in the second unit 1208, and particularly from stabilization bays 1218 to treatment cubicles 1222.

Although not explicitly shown in FIGS. 12-14, the graphical user interface can incorporate an augmented reality overlay (e.g., a two-dimensional or three-dimensional model of units 302) superimposed over images of the units 302 of the hospital (e.g., the images can be captured by a camera, such as a smart phone, of the units 302).

It should be appreciated that, although FIGS. 12-14 illustrates an example visualization of a simulated scenario for purposes of configuration and user interaction, the simulation component 118 is not required to visually render or animate the simulation during execution. In various embodiments, the simulation component 118 can execute the simulation solely to generate simulation data 108 (e.g., event logs, state transitions, performance metrics, statistical outputs) without producing a graphical depiction of patient movement, resource usage, or system dynamics. In such embodiments, visualization of the simulation, as depicted in FIGS. 12-14, can be omitted, deferred, or selectively enabled, while the generated simulation data 108 is provided to analysis component 120 for detecting operational bottlenecks 124 and generating action plan recommendations 106, data summary 702, and data visuals 704.

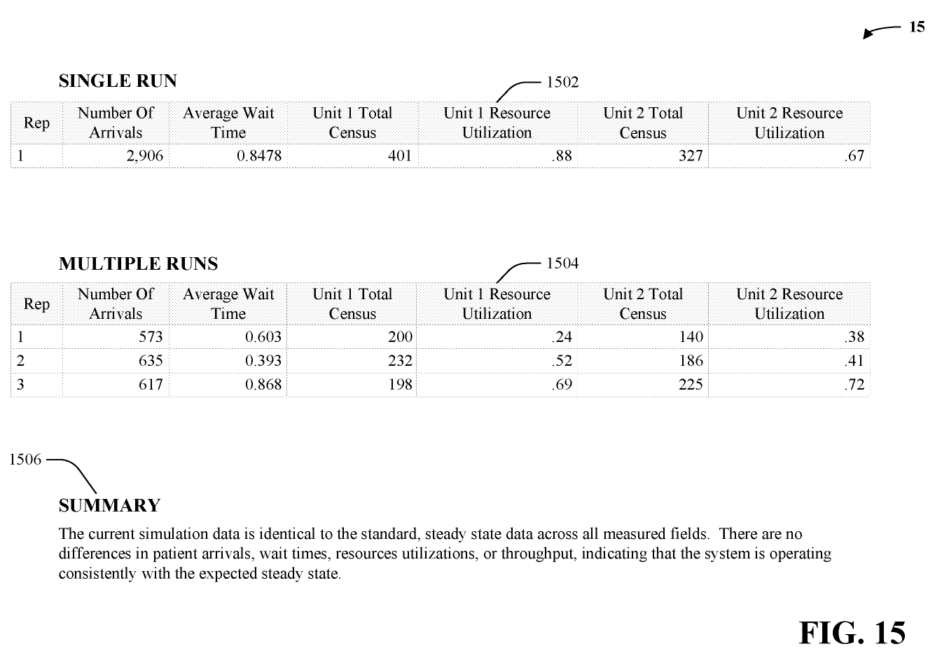

FIG. 15 illustrates a diagram of an example, non-limiting GUI 1500 showing simulation data and a simulation summary in accordance with one or more embodiments described herein.

In various embodiments, the simulation component 118 can run a simulation of a scenario in a hospital any suitable number of times. For example, simulation component 118 can simulate the scenario one time, and in other instances can simulate the scenario multiple times. In various cases, the number of repetitions to simulate the scenario can be specified by a user and received as input data 114.

In various aspects, the simulation component 118 can generate simulation data 1502 for a single repetition of the simulation. In various instances, the simulation component 118 can generate simulation data 1504 for multiple repetitions of the simulation. That is, the simulation component 118 can perform any suitable number of repetitions of the simulation (e.g., using configuration 104 and/or alternate configurations 802) to generate simulation data 1504, wherein the simulation data 1504 comprises data for each repetition. As shown in the non-limiting example of FIG. 15, simulation data 1504 is generated for 3 repetitions of the simulation.

In various aspects, the simulation data 1502 and the simulation data 1504 can include data for various metrics of the simulation (e.g., average patient wait times for each queue, average time a cubicle is occupied by a patient, total number of patients that arrived). As a non-limiting example, the simulation data 1502 and the simulation data 1504 includes number of arrivals, average wait time, and resource utilization of each unit.

For instance, in simulation data 1502 for one repetition, 2,906 patients arrived at the emergency department 1202 over the duration of the simulation. As another example, in simulation data 1504 for multiple repetitions, 573, 635, and 617 patients arrived at the emergency department in the first, second, and third repetition, respectively.

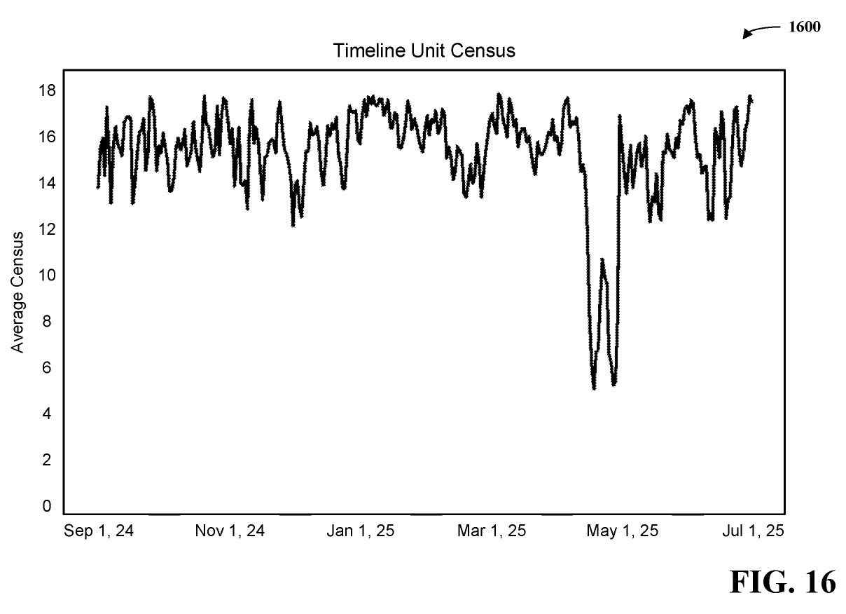

In various cases, simulation data 1502 voluminous, multi-dimensional, and/or technically intricate, thereby rendering direct interpretation of the simulation data 1502 by an end-user difficult. Accordingly, the analysis component 120 can generate, via the LLM 204, a summary 1506 of simulation data 1502 and/or simulation data 1504. For example, summary 1506 can state “The current simulation data is identical to the standard, steady state data across all measured fields. There are no differences in patient arrivals, wait times, resources utilizations, or throughput, indicating that the system is operating consistently with the expected steady state.” By generating a summary 1506 of the simulation data, an end-user (e.g., healthcare administrators, staff) can be provided with user-friendly and digestible information regarding the resources of the hospital and the efficiency of their resource utilization. In various cases, the analysis component 120 can also generate, via the LLM 204, data visuals 704 based on the simulation data 1502 and/or simulation data 1504 to further aid an end-user in understanding the simulation results. Various non-limiting examples of data visuals bare described with respect to FIGS. 16-26.

Although the various embodiments described herein primarily describe simulation and emulation of an emergency department, hospital simulation and emulation can be performed for any suitable medical unit, department, hospital, or clinical organization. As non-limiting examples, the various embodiments described herein can facilitate simulation and emulation of an outpatient clinic, a cardiology department, a pharmacy, a pediatric department, a dermatology department, or a rehabilitation department. Likewise, although the various embodiments described herein primarily describe simulation and emulation of three units within an emergency department, hospital simulation and emulation can be performed for any suitable number or type of medical units, departments, hospitals, or clinical organizations. In some instances, the various embodiments described herein can facilitate simulation and emulation of two or more departments comprising one or more units.

FIGS. 16-26 illustrate example, non-limiting data visuals and data summaries in accordance with one or more embodiments described herein. As described elsewhere, analysis component 120 can generate, via LLM 204, data summary 702 and data visuals 704 based on simulation data 108. Thereafter, display component 402 can render, via GUI 404, the data summary 702 and data visuals 704 on any suitable electronic display. In various aspects, the data summary 702 and data visuals 704 can also identify, describe, or otherwise convey the operational bottlenecks 124 and action plan recommendations 106. Accordingly, the data summaries and data visuals depicted in FIGS. 16–26 can be generated by executing LLM 204 on simulation data (e.g., simulation data 108) to transform complex, granular, or technical hospital simulation outputs into human-readable explanations and intuitive visual representations, thereby facilitating comprehension of operational performance, trends, and resource constraints by technical and non-technical stakeholders such as clinicians, administrators, or operational personnel. Further, the data summaries and data visuals depicted in FIGS. 16–26 can be generated based on detected operational bottlenecks (e.g., operational bottlenecks 124) and generated action plan recommendations (action plan recommendations 106).

FIG. 16 illustrates an example, non-limiting data visual 1600 in accordance with one or more embodiments described herein. In particular, data visual 1600 comprises a graph representing changes in a hospital unit census over a period of time. The horizontal axis (x-axis) corresponds to discrete or continuous dates or time intervals, while the vertical axis (y-axis) represents an average number of patients admitted to beds allocated within a specific hospital unit. In various aspects, the values depicted along the y-axis can be derived from simulation data reflecting bed occupancy, patient admissions, discharges, or transfers associated with the unit. As such, the data visual 1600 can generally represent a number of occupied beds, wherein an occupied bed corresponds to a bed to which a patient is admitted at a given time.

By visually depicting census fluctuations over time, the data visual 1600 can enable users to identify utilization patterns, peak occupancy periods, and potential capacity constraints, which can correspond to operational bottlenecks 124 identified by the analysis component 120. In some embodiments, the data visual 1600 can be presented in conjunction with a natural-language data summary describing observed trends, anomalies, or recommended actions (e.g., action plan recommendations 106) for improving unit-level operational efficiency.

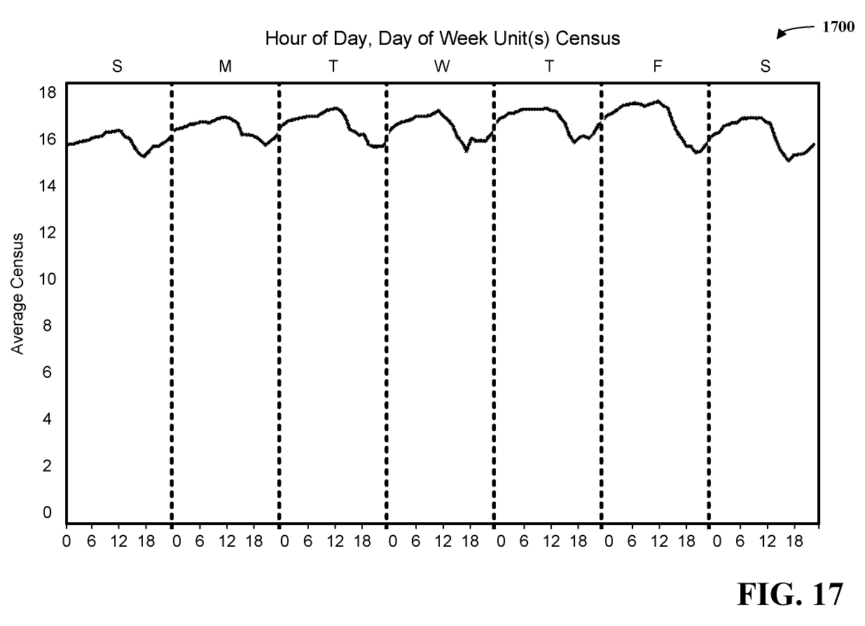

FIG. 17 illustrates an example non-limiting data visual 1700 in accordance with one or more embodiments described herein. In particular, data visual 1700 comprises a graph representing changes in a hospital unit census by hour of the day and day of the week.

The horizontal axis (x-axis) represents the hour of the day and the day of the week (e.g., over one week), and the vertical axis (y-axis) represents an average number of patients admitted to beds allocated within a specific hospital unit at the top of each hour. In various aspects, the plotted values can be derived from simulation data reflecting hourly admission, discharge, and transfer activity. The data visual 1700 can thereby illustrate intra-day and inter-day census variability, enabling users to identify recurring temporal patterns, such as predictable peaks or troughs in occupancy, which can be indicative of inefficient resource utilization (e.g., staffing misalignment, capacity underutilization) or operational bottlenecks within the unit.

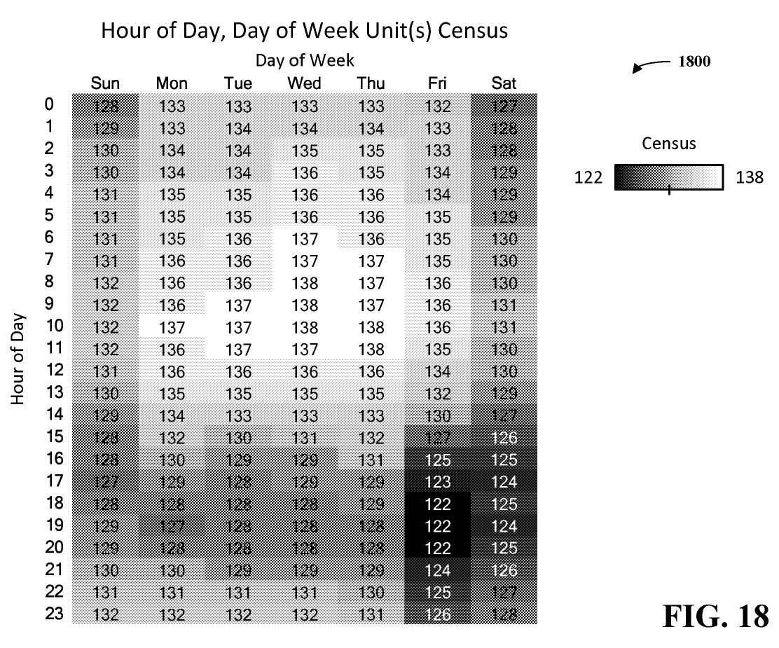

FIG. 18 illustrates an example non-limiting data visual 1800 in accordance with one or more embodiments described herein. In particular, data visual 1800 comprises a heat map representation of a hospital unit’s census over one week. The horizontal axis (x-axis) represents the day of the week, and the vertical axis (y-axis) represents the hour of the day. Each cell or entry within the heat map corresponds to a specific day-hour combination and is visually encoded using a color or shading value that reflects a number of beds occupied by patients in the unit during that interval. In the heat map, a darker shading (e.g., black or near-black) indicates lower occupancy levels and greater bed availability, whereas lighter shading (e.g., white or near-white) indicates higher occupancy levels and reduced availability. By aggregating and visually encoding simulation data 108 in this manner, the data visual 1800 can enable rapid identification of temporal congestion patterns, sustained high-utilization periods, and operational bottlenecks. For instance, lighter shading can indicate that higher patient occupancy can result in delayed patient admissions or throughput constraints.

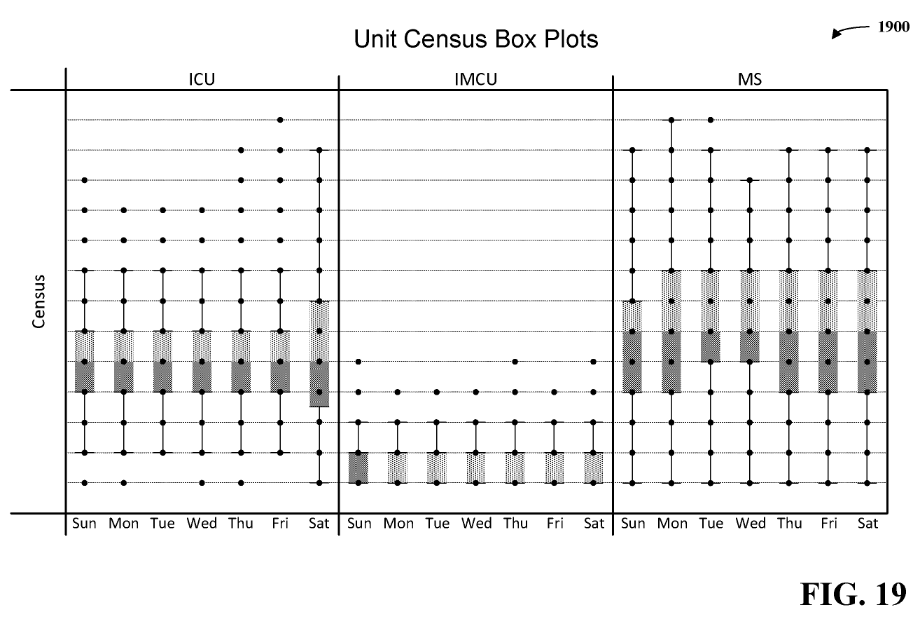

FIG. 19 illustrates an example non-limiting data visual 1900 in accordance with one or more embodiments described herein. In particular, data visual 1900 comprises a set of box plots corresponding to multiple hospital units (e.g., ICU, IMCU, and MS). For each box plot, the horizontal axis (x-axis) represents the day of the week, and the vertical axis (y-axis) represents hospital unit census (e.g., the number of occupied beds within the respective unit). Each box plot graphically represents the census data distribution, including a minimum value, a first quartile, a median, a third quartile, and a maximum value, thereby conveying variability and dispersion in census levels. By presenting box plots for multiple units in a side-by-side arrangement, the data visual 1900 facilitates comparative analysis across units, enabling users to identify differences in utilization patterns, volatility, and peak occupancy. Additionally, the data visual 1900 can illustrate day-to-day and week-to-week census variation, which can be indicative of operational bottlenecks and inefficiencies or capacity imbalances among the units.

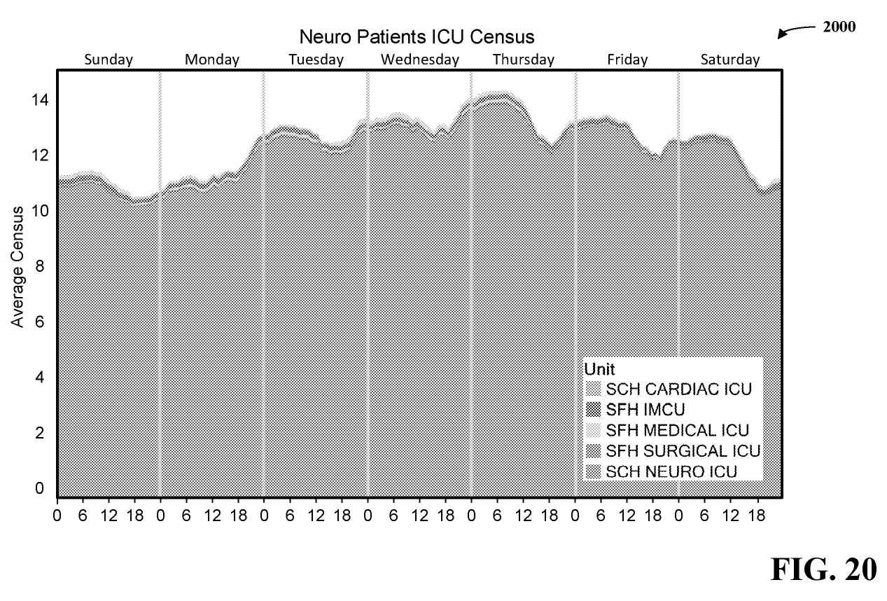

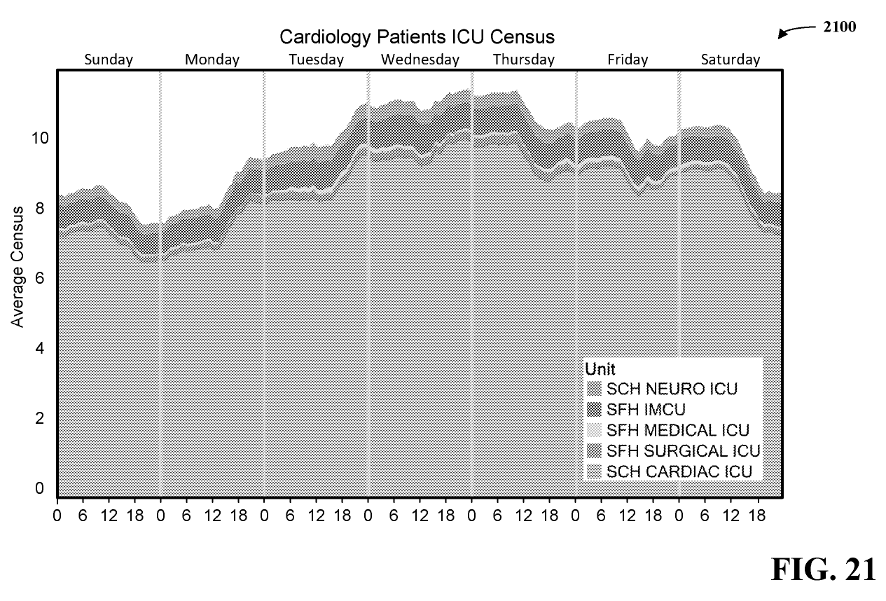

FIGS. 20 and 21 illustrate example non-limiting data visuals 2000 and 2100 in accordance with one or more embodiments described herein. In particular, data visual 2000 and data visual 2100 each comprise a graph representing a hospital unit’s census segmented by patient type. The horizontal axis (x-axis) represents the hour of the day, and the vertical axis (y-axis) represents the average census. In various aspects, the graphs illustrate census trends over time for different categories of patients admitted to the same unit, such as cardiac patients, medical patients, surgical patients, and neurological patients concurrently admitted to the ICU. By disaggregating census data by patient type, the data visual 2000 and data visual 2100 can enable users to assess relative contributions of different clinical populations to overall unit occupancy, identify patient-type-specific surges, and evaluate the impact of case mix on capacity utilization and throughput.

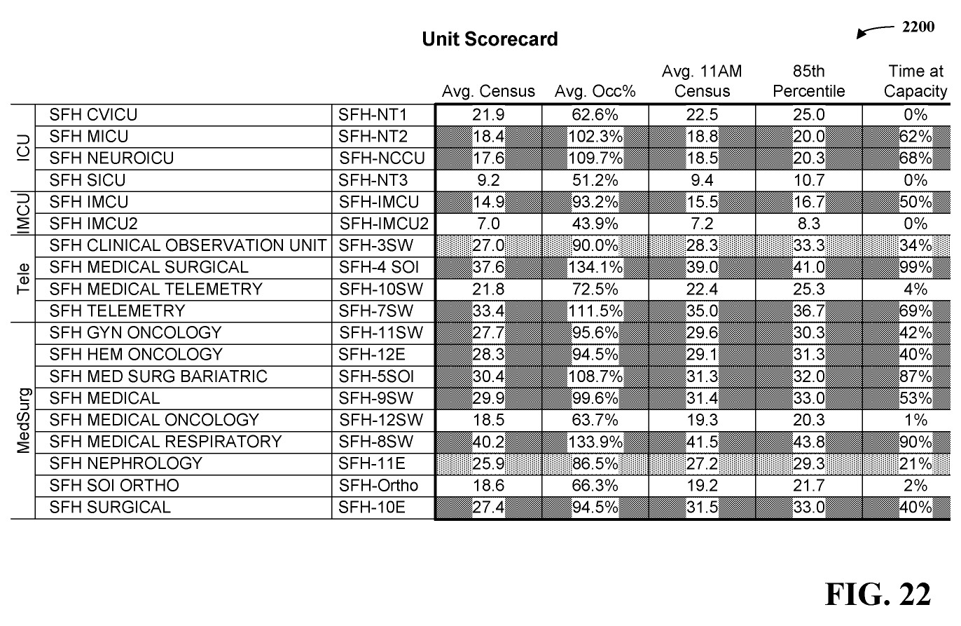

FIG. 22 illustrates an example non-limiting data visual 2200 in accordance with one or more embodiments described herein. In particular, data visual 2200 comprises a scorecard visual representation of operational metrics for a plurality of hospital units. The scorecard can list multiple types of census-related computations and performance indicators for each unit. Non-limiting examples of such metrics include average census, average occupancy, average census at a predetermined time (e.g., 11:00 a.m.), an 85th percentile census value, a proportion of time at or near capacity, and other statistically derived or operationally relevant measures. In various aspects, the data visual 2200 can be dynamically generated from simulation data and can be configurable or customizable by an end user to include selected metrics, thresholds, or scoring criteria. Such a scorecard can support rapid comparison of unit performance under different simulated scenarios, configurations, or operational conditions.

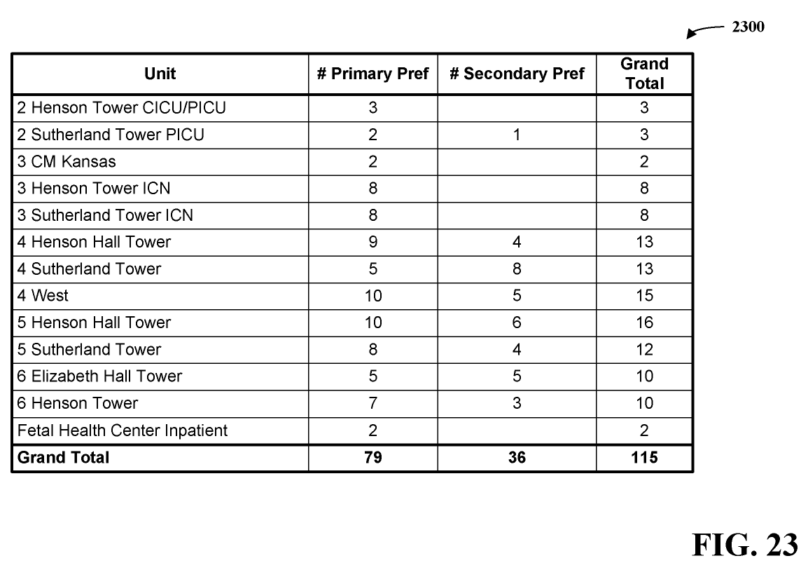

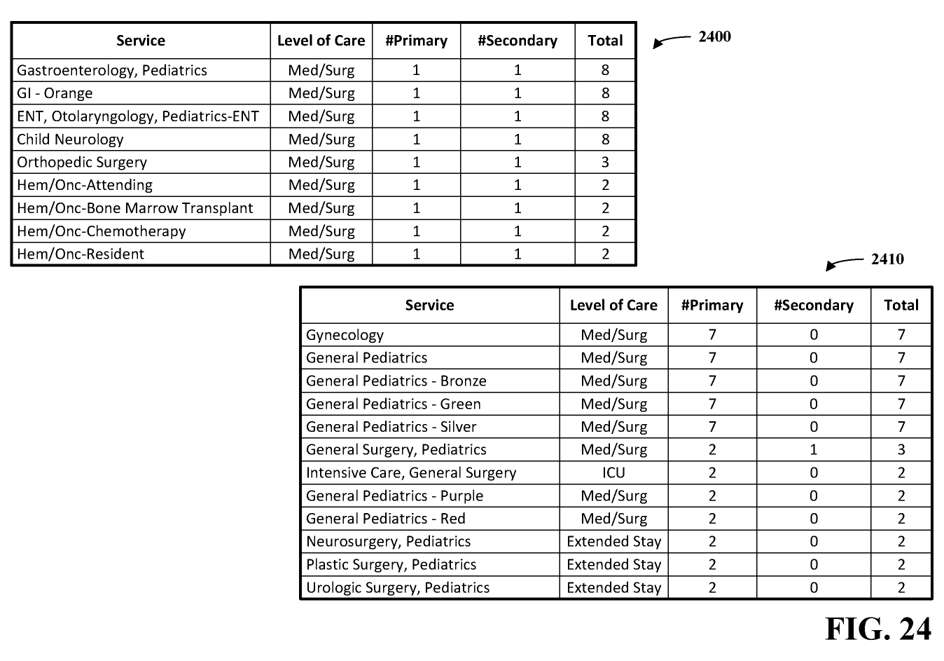

FIGS. 23 and 24 illustrate example non-limiting data visuals 2300, 2400, and 2410 in accordance with one or more embodiments described herein. In particular, data visual 2300 comprises a chart depicting counts of clinical service preferences including a number of services designating each unit as a primary preferred admission destination (denoted as “# primary pref”), a number of services designating each unit as a secondary preferred admission destination (denoted as “# secondary pref”), and a corresponding grand total for a plurality of units.

Data visual 2400 and data visual 2410 each comprise a chart depicting how the services are allocated to primary or secondary units. In particular, the data visuals 2400 and 2410 illustrate, for a plurality of clinical services, an associated level of care and corresponding counts of primary preferred admission destinations (denoted as “# primary”), and secondary preferred admission destinations (denoted as “# secondary”) across hospital units. In various aspects, these visuals can enable users to evaluate how individual services are allocated to primary and secondary units at different levels of care, identify fragmentation or dispersion of service assignments, and assess opportunities to consolidate or realign service-to-unit mappings. By presenting preference metrics at both the unit level (data visual 2300) and the service level (data visuals 2400 and 2410), analysis of service scatter, capacity alignment, and operational optimization across the hospital can be enabled.

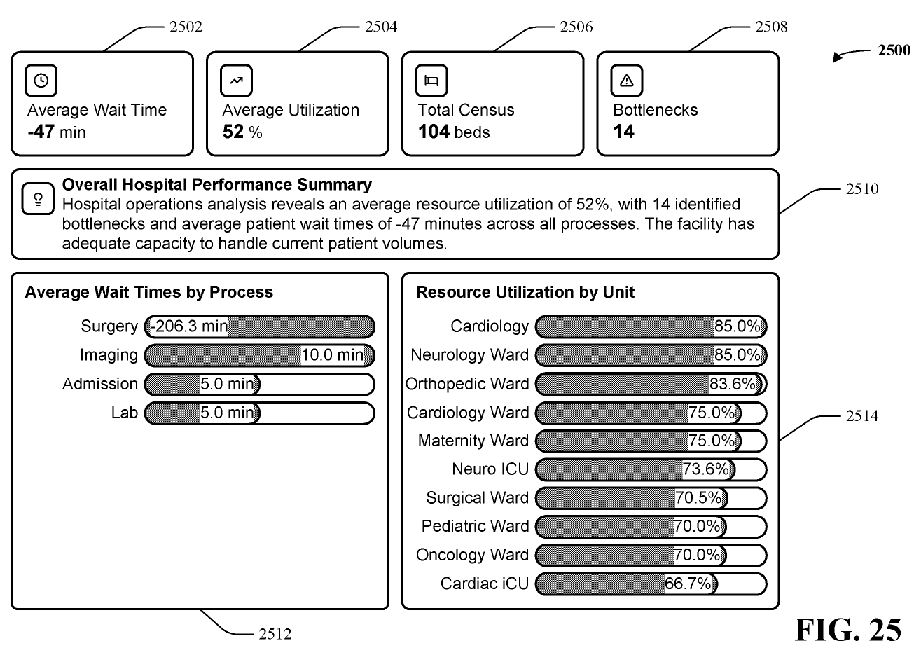

FIG. 25 illustrates an example non-limiting GUI 2500 showing data visuals and data summaries in accordance with one or more embodiments described herein. In particular, GUI 2500 can comprise a dashboard interface presenting a consolidated, high-level summary of simulation data (e.g., simulation data 108) for one or more hospital units or departments. The dashboard can include a plurality of visual elements or metric panels configured to convey key operational performance indicators. For example, the dashboard can display an average patient wait time (depicted in box 2502), an average resource utilization metric (depicted in box 2504), a total census (depicted in box 2506), and the number of operational bottlenecks 124 that are detected (depicted in box 2508). In various instances, the average resource utilization can be computed as a ratio of time during which a resource is actively used relative to a total time period during which the resource is available, and can be expressed as a percentage.

The dashboard can further include a data summary 2510 generated via LLM 204 based on simulation data 108. In various instances, the data summary 2510 can describe overall unit performance, highlight resource utilization trends, and identify significant operational bottlenecks or resource constraints requiring attention. For example, as shown in FIG. 25, the data summary 2510 can state that hospital operations analysis reveals an average resource utilization of approximately 52 percent, a plurality of identified bottlenecks (e.g., 14 bottlenecks), and average patient wait times of approximately negative 47 minutes across simulated processes, thereby indicating that the unit has sufficient capacity to accommodate current patient volumes.

The dashboard can further include average wait times segmented by process or procedure (depicted in box 2512), such as by surgery, imaging, admission, or laboratory procedures. Additionally, the dashboard can include the resource utilization for a plurality of units or subunits (depicted in box 2514), such as for cardiology, neurology, and orthopedic units or subunits, thereby enabling more granular assessment of utilization patterns within each hospital unit.

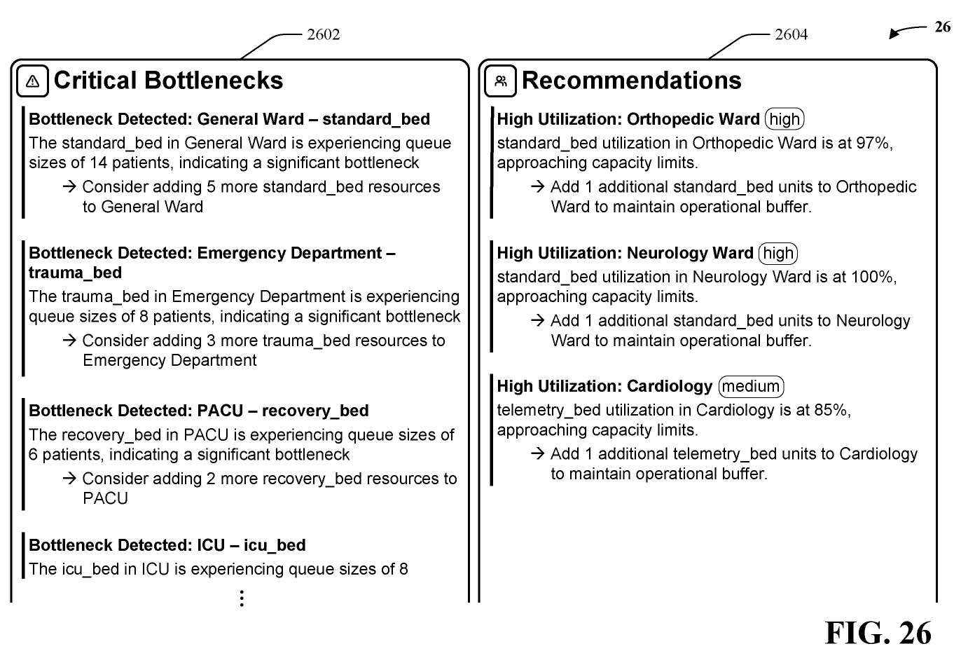

FIG. 26 illustrates an example non-limiting GUI 2600 showing data visuals and data summaries in accordance with one or more embodiments described herein. In particular, GUI 2600 comprises a dashboard interface presenting operational bottlenecks 124 that are detected and corresponding action plan recommendations 106 for one or more hospital units or departments. Specifically, the dashboard can present a visual listing or categorization of operational bottlenecks 124 (depicted in box 2602) and corresponding action plan recommendations 106 (depicted in box 2604). The operational bottlenecks can include, for example, resource shortages or constraints such as insufficient bed capacity, limited availability of operating rooms, shortages of specialized clinical staff (e.g., trauma surgeons), or other factors that contribute to delays or unavailability of patient care processes. The action plan recommendations 106 can describe corrective actions or operational adjustments to mitigate the operational bottlenecks 124, such as reallocating resources, modifying staffing levels or schedules, adjusting admission or discharge workflows, or reconfiguring unit capacities. In various aspects, the GUI 2600 can present the operational bottlenecks 124 and action plan recommendations 106 in a structured, prioritized, or ranked format to facilitate rapid assessment and decision-making by users. By visually linking operational bottlenecks 124 with action plan recommendations 106, the GUI 2600 can support proactive operational planning and continuous improvement of hospital performance.

It should be appreciated that the data visuals and data summaries illustrated in FIGS. 16–26 are provided as non-limiting examples, and that, in various embodiments, any suitable combination of graphical representations, dashboards, metrics, charts, tables, and natural-language summaries can be generated and presented to facilitate interpretation of simulation data, identification of operational trends and operational bottlenecks, and informed decision-making regarding hospital operations and resource optimization through action plan recommendations.

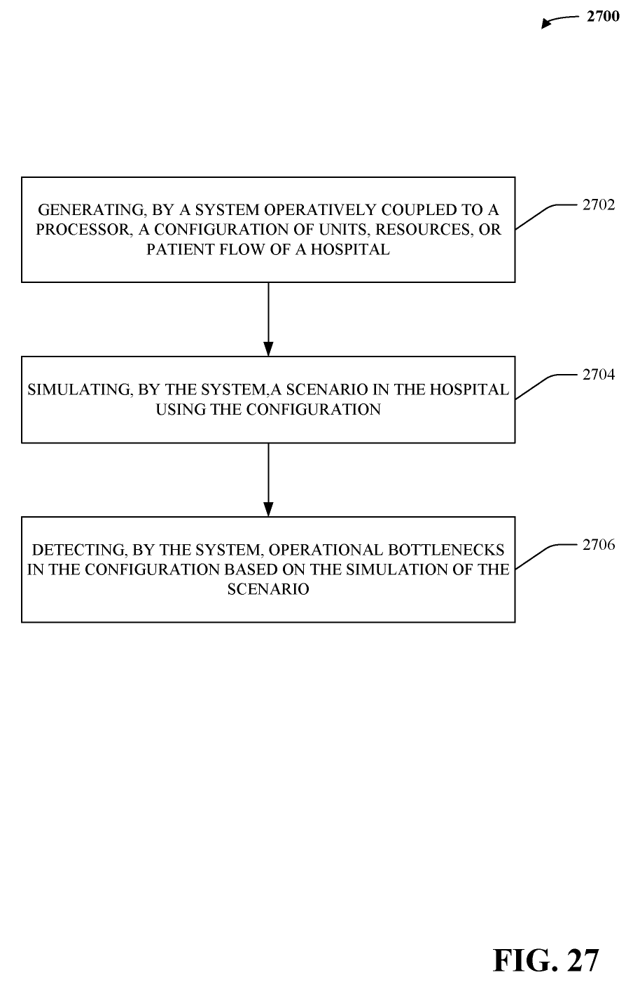

FIG. 27 illustrates a flow diagram of an example, non-limiting computer-implemented method 2700 that can facilitate intelligent hospital scenario simulation and emulation in accordance with one or more embodiments described herein. In various cases, the hospital operations optimization system 102 can facilitate the computer-implemented method 2700.

In various embodiments, act 2702 can include generating, by a system (e.g., via modeling component 116) operatively coupled to a processor (e.g., 110), a configuration (e.g., 104) of units (e.g., 302), resources (e.g., 304), or patient flow (e.g., 306) of a hospital.

In various aspects, act 2704 can include simulating, by the system (e.g., via simulation component 118), a scenario in the hospital using the configuration.

In various instances, act 2706 can include detecting, by the system (e.g., via analysis component 120), operational bottlenecks (e.g., 124) in the configuration based on the simulation of the scenario.

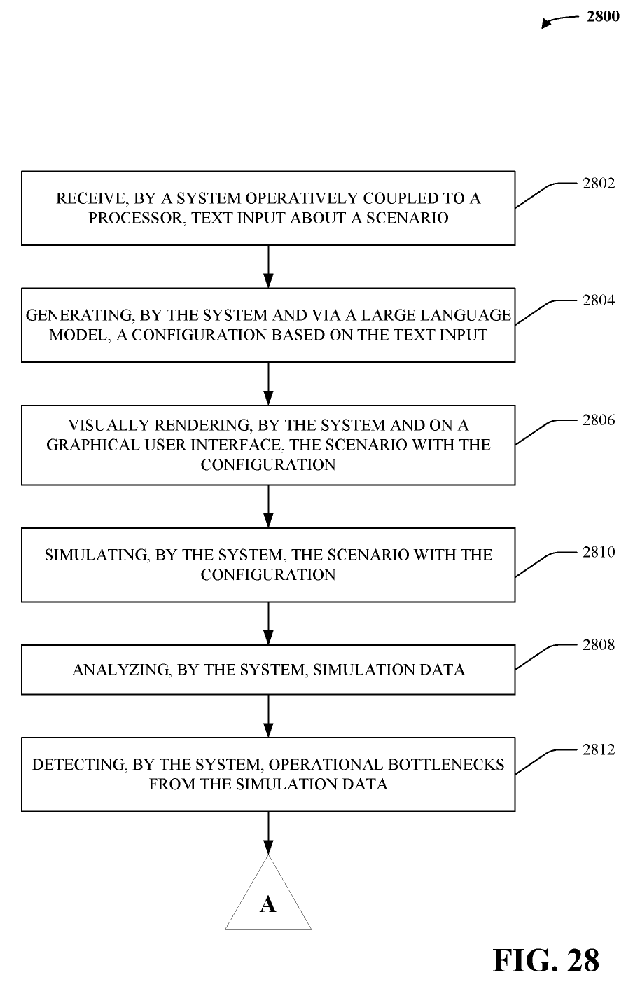

FIG. 28 illustrates a flow diagram of an example, non-limiting computer-implemented method 2800 that can facilitate intelligent hospital scenario simulation and emulation in accordance with one or more embodiments described herein. In various cases, the hospital operations optimization system 102 can facilitate the computer-implemented method 2800.

In various embodiments, act 2802 can include receiving, by a system (e.g., via modeling component 116) operatively coupled to a processor (e.g., 110), text input about a scenario in a hospital.

In various aspects, act 2804 can include generating, by the system (e.g., via modeling component 116) and via an LLM (e.g., 202), a configuration based on the text input.

In various instances, act 2806 can include visually rendering, by the system (e.g., via display component 402) and on a graphical user interface (e.g., 404), the scenario with the configuration.

In various aspects, act 2808 can include simulating, by the system (e.g., via simulation component 118), the scenario with the configuration. Such simulation can result in the generation of simulation data.

In various aspects, act 2810 can include analyzing, by the system (e.g., via analysis component 120), the simulation data.

In various aspects, act 2812 can include detecting, by the system (e.g., via analysis component 120), operational bottlenecks from the simulation data.

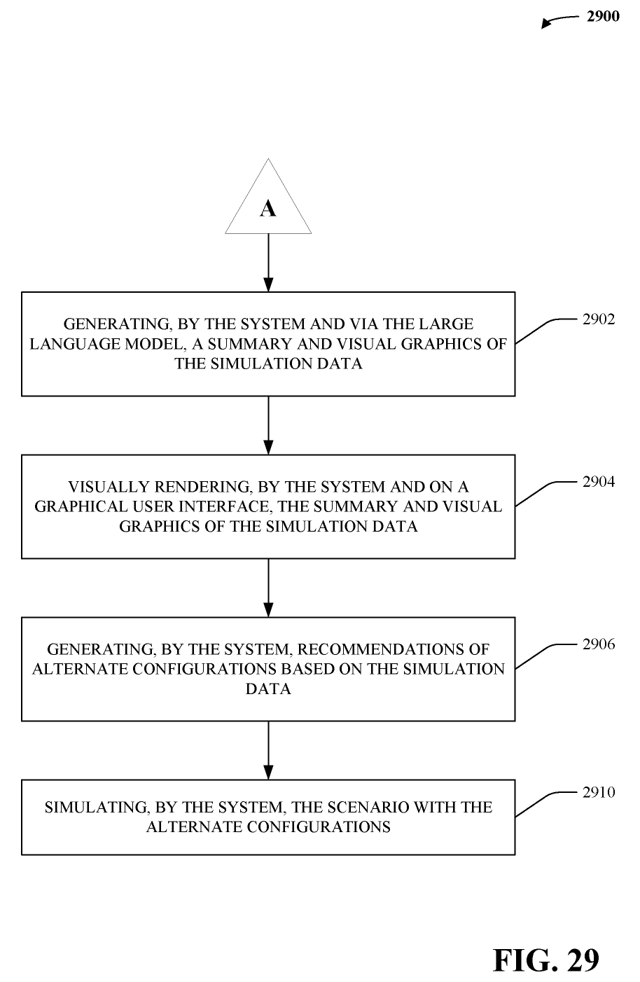

FIG. 29 illustrates a flow diagram of an example, non-limiting computer-implemented method 2900 that can facilitate intelligent hospital scenario simulation and emulation in accordance with one or more embodiments described herein. In various cases, the hospital operations optimization system 102 can facilitate the computer-implemented method 2900.

In various embodiments, act 2902 can include generating, by a system (e.g., via modeling component 116) operatively coupled to a processor (e.g., 110) and via an LLM (e.g., 202), a summary and visual graphics of the simulation data.

In various aspects, act 2904 can include visually rendering, by the system (e.g., via display component 402) and on a graphical user interface (e.g., 404), the summary and visual graphics of the simulation data.

In various instances, act 2906 can include generating, by the system (e.g., via analysis component 120), recommendations of alternate configurations based on the simulation data.

In various instances, act 2908 can include simulating, by the system (e.g., via simulation component 118), the scenario with the alternate configurations.

Although various embodiments are described herein with respect to GUIs, this is a mere non-limiting example for ease of explanation and illustration. In various other embodiments, the teachings described herein can be applied or extrapolated to any suitable electronic user interfaces (e.g., are not limited only to graphical user interfaces). As a non-limiting example, various embodiments described herein can be applied or implemented to an audio user interface.

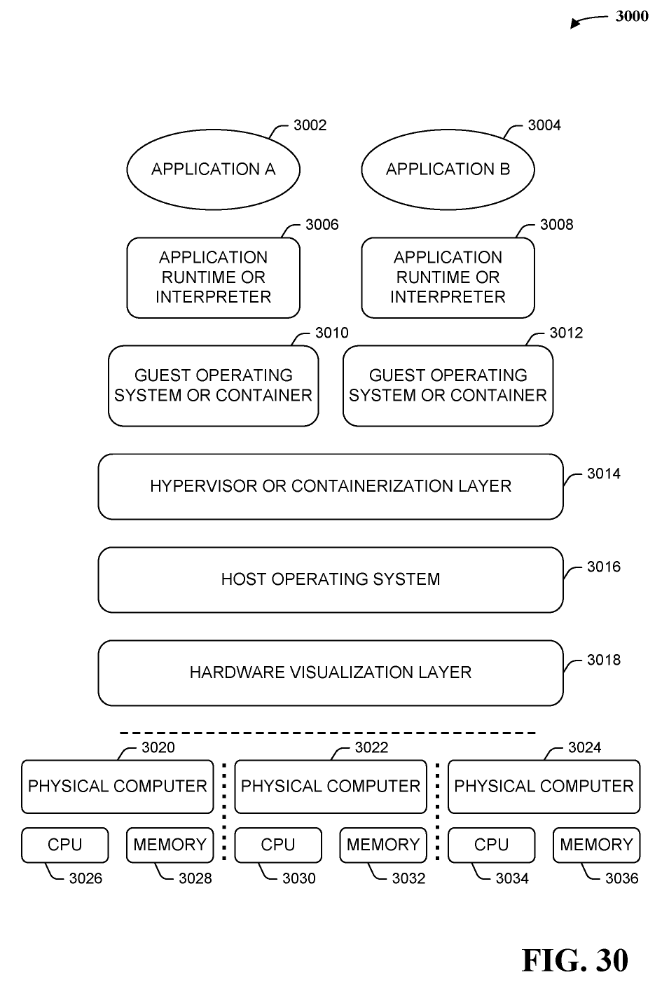

FIG. 30 illustrates a block diagram of an example, non-limiting computing architecture 3000 in which one or more embodiments described herein can be facilitated.

The computing architecture 3000 depicts a layered execution model commonly employed in cloud and virtualized computing environments. In the illustrated embodiment, one or more applications (e.g., application A 3002 and application B 3004) can execute within a corresponding application runtime or interpreter (e.g., application runtime or interpreter 3006, 3008). Non-limiting examples of such runtimes or interpreters include virtual machines, language runtimes, or execution environments for programming languages such as Java, Python, or JavaScript.

The application runtime or interpreter can execute within either a guest operating system or a containerized environment (e.g., guest operating systems or containers 3010, 3012). In some embodiments, containerized environments can execute atop a containerization layer, whereas guest operating systems can execute atop a hypervisor layer. As shown, a hypervisor or containerization layer 3014 can abstract underlying system resources and provide isolation between multiple execution environments. The hypervisor or containerization layer 3014 can execute on a host operating system 3016, which can include, for example, Linux, Windows, UNIX, or other suitable operating systems.

The host operating system 3016 can execute on a hardware virtualization layer 3018, which abstracts underlying physical computing resources. The hardware virtualization layer 3018 can present a unified, virtualized view of hardware resources (e.g., processors, memory, buses, and input/output devices) to the host operating system 3016, regardless of the specific type, configuration, or capacity of the underlying hardware. As illustrated, the hardware virtualization layer 3018 can be backed by one or more physical computing devices (e.g., physical computers 3020, 3022, 3024), each comprising physical processors (e.g., CPUs 3026, 3030, 3034) and memory components (e.g., memory 3028, 3032, 3036).

In various embodiments, processing operations described herein can be executed by one or more processors. The processor can include a physical processor, a virtual processor provisioned by a virtualization infrastructure, or processing resources provided by a cloud computing service that presents a virtual layer of processors. The virtual layer of processors can be implemented through hypervisor-based virtualization, container orchestration frameworks, or distributed computing infrastructures that allocate processing resources across one or more physical computing devices.

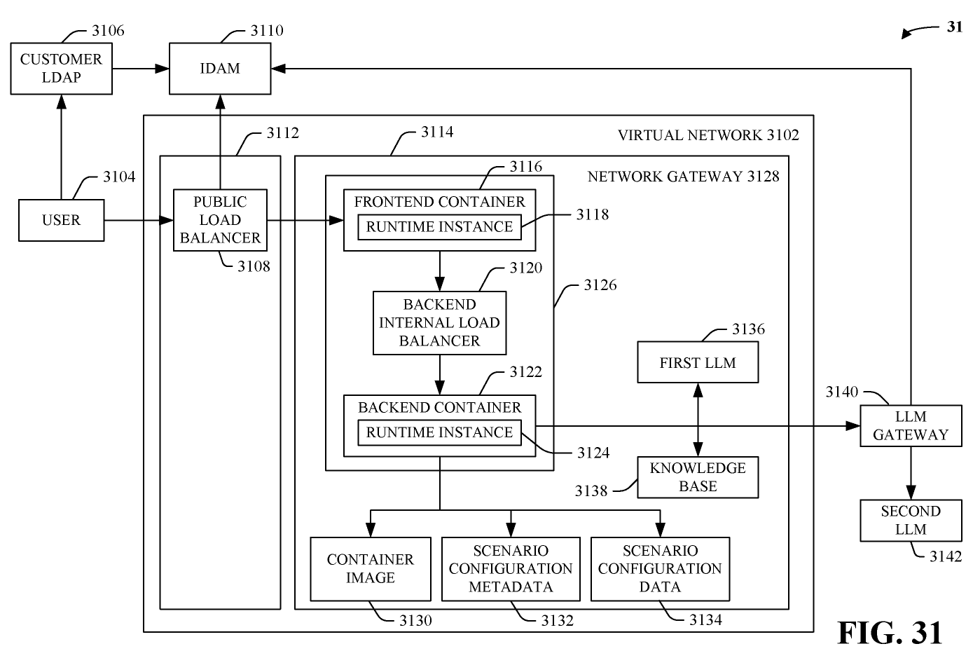

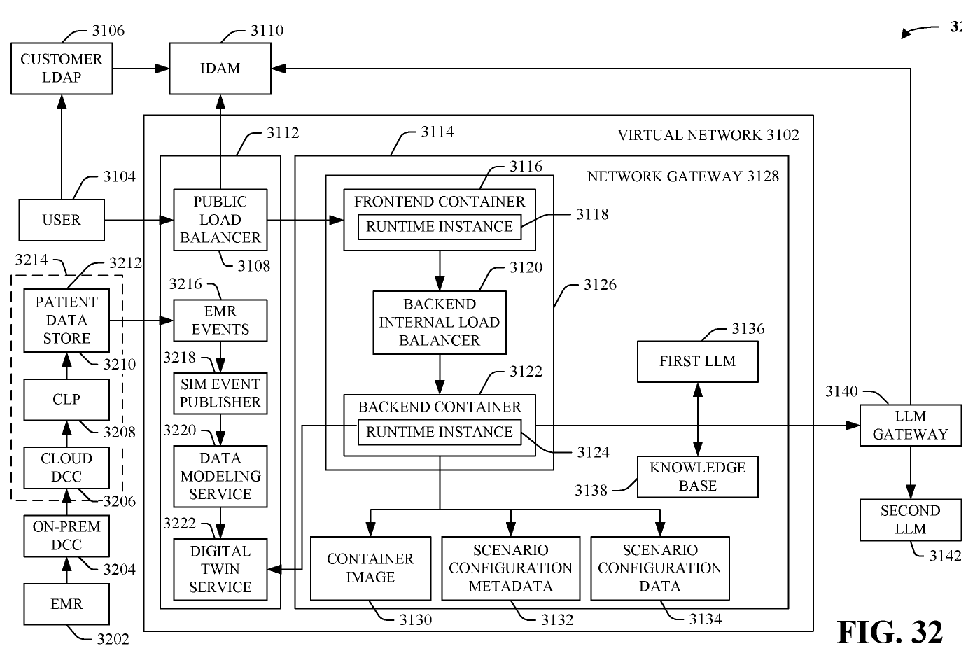

In operation, applications executing within the computing architecture 3000 can perceive a single, consistent operating system environment and a set of virtualized hardware resources, even though the actual execution may be distributed across heterogeneous physical computing devices. This abstraction enables scalable, portable, and isolated execution of applications and services, including the containerized workloads, runtime instances, simulations, large language model interactions, and components described with respect to FIGS. 31 and 32.

FIGS. 31 and 32 illustrate block diagrams of an example, non-limiting system architectures 3100 and 3200 in which one or more embodiments described herein can be facilitated.

In particular, non-limiting system architecture 3100 can facilitate an off-the-shelf Software-as-a-Service (SaaS) platform to enable intelligent hospital scenario simulation and emulation.

In various aspects, a virtual network 3102 can be segmented into public subnet 3112 and private subnet 3114. In various aspects, resources operating within the private subnet 3114 can use a network gateway 3128 to enable secure outbound connectivity.

In various aspects, a user 3104 can authenticate via Customer LDAP 3106, after which an administrator can provision access through an IDAM 3110 by assigning group-based permissions. When the user 3104 accesses the application endpoint, a public load balancer 3108 on public subnets 3112 can validate the IDAM-minted token and can redirect to a federation page of IDAM 3110 if a valid token is not present. With a valid OIDC token, the public load balancer 3108 can forward the request into a frontend container 3116, executing within a runtime instance 3118 on a container orchestration cluster 3126 in private subnet 3114. The frontend container 3116 can then retrieve scenario configuration metadata 3132 from metadata storage and can identify the corresponding path in object storage to load the scenario definition (scenario configuration data 3134). In some cases, the user 3104 can modify the scenario and can save it under a new version or name, where the scenario configuration metadata 3132 and scenario configuration data 3134 is updated and saved in metadata storage and object storage. The frontend container 3116 and backend container 3122, along with their runtime instances 3118 and 3124, can operate within the container orchestration cluster 3126.

When the user 3104 initiates a simulation, the frontend container 3116 can prepare the simulation configuration (e.g., configuration 104) and can call backend services through a backend internal load balancer 3120. The backend internal load balancer 3120 can distribute requests to one or more backend containers 3122, each executing within a runtime instance 3124 of the container orchestration cluster 3126 in the private subnet 3114. The backend containers 3122 can execute the simulation, can save scenario configuration data 3134 to object storage, and can perform post-processing, including summarization guided by scenario configuration metadata 3132. Backend containers 3122 can further integrate with a first LLM 3136 (e.g., LLM 204) via an LLM gateway 3140 and can access a knowledge base 3138 for retrieval-augmented generation to generate data summaries (e.g., data summary 702) and recommended operational improvements (e.g., action plan recommendations 106). The first LLM 3136 can be a trained language model configured to generate intermediate outputs, such as structured summaries, interpretations, extracted insights, or transformed representations of simulation data. Outputs generated by the first LLM 3136 can be provided, in whole or in part, as prompts, contextual inputs, or intermediate artifacts to a second LLM 3142, which can further process the intermediate outputs to generate refined results, such as data summaries (e.g., data summary 702), detected operational bottlenecks (e.g., operational bottlenecks 124), or recommended operational improvements (e.g., action plan recommendations 106).

In various aspects, the runtime instance 3124 can orchestrate a sequence of processing steps and dynamically determine which large language model to invoke at each step (e.g., first LLM 3136 or second LLM 3142) depending on the nature of the task to be performed. In various aspects, first LLM 3136 and second LLM 3142 can be trained on different datasets. Processed analysis results can be written to object storage and updated scenario configuration data 3134 can be recorded in metadata storage. The frontend container 3116 can retrieve the updated information and can render findings, analysis, and recommendations to the user 3104 (e.g., render data summary 702, data visuals 704, operational bottlenecks 124, alternate configuration 802, and/or action plan recommendations 106 via GUI 404).

A similar process can be executed when the user 3104 wants to model a hypothetical scenario with AI assistance. In this case, the frontend container 3116 can fetch scenario configuration metadata 3132 from metadata storage and can initiate an interaction with the first LLM 3136 to generate a new scenario. The output generated by the first LLM 3136 can be supplied as an input prompt to the second LLM 3142, which can further refine the scenario. The output of the second LLM 3142 can be returned to the runtime instance 3124, which can determine subsequent action items, including invoking additional LLMs, executing functions or tools, persisting scenario data, or triggering simulation execution by the backend containers 3122. The LLM output (e.g., from first LLM 3136 and/or second LLM 3142) can be parsed to execute functions or tools to save the scenario before the frontend container 3116 can call the backend containers 3122 to execute the simulation, analyze the data, and produce summaries and recommendations in the manner described above.

The non-limiting system architecture 3200 can facilitate a real-time, data-integrated SaaS platform to enable intelligent hospital scenario simulation and emulation. In various aspects, components 3202 through 3222 can collectively operate to ingest, extract, normalize, transform, and populate a dataset derived from hospital operational data for downstream modeling and analysis. In the non-limiting system architecture 3200, an EMR system 3202 (e.g., a commercially available EMR platform maintained by a hospital) can generate clinical and operational data, including structured records and unstructured natural-language content. An on-premises data connectivity component (on-prem DCC) 3204 can securely extract data from the EMR system 3202 and can forward the data to a cloud-based data connectivity component (cloud DCC) 3206. The cloud DCC 3206 can transfer data to a clinical logic processor (CLP) 3208. CLP 3208 can perform clinical language processing and data enrichment operations, such as extracting medical terms, clinical concepts, or other semantically meaningful information from unstructured or semi-structured EMR content (e.g., physician notes or narrative documentation). Such extracted concepts can enable downstream systems to operate on information that would otherwise be inaccessible or unusable in raw textual form. The resulting patient-related data can be persisted to a patient data store 3212 within a cloud environment 3214. In some embodiments, the patient data store 3212 can function as a required store-and-forward component within an existing customer architecture.

The patient data store 3212 can export data as one or more data streams (e.g., HL7, FHIR) represented as EMR events 3216. In various embodiments, the EMR events 3216 can be transmitted via a streaming service operating within a private subnet 3114 of the virtual network 3102. A simulation event publisher 3218, which can be implemented using a serverless or managed compute service, can receive the streamed EMR events and perform filtering, mapping, and transformation operations. In particular, the simulation event publisher 3218 can convert EMR-derived data into a format required by a downstream data modeling service 3220 (e.g., an IoT-based data modeling service), thereby bridging incompatibilities between EMR interoperability standards and the input requirements of the modeling service.

The data modeling service 3220 can organize, normalize, and structure the transformed data into a schema consumable by a digital twin service 3222 executing within the private subnet 3114. The digital twin service 3222 can ingest the structured data to represent operational entities, processes, or system states within the hospital environment. In this architecture, components 3202 through 3222 primarily operate to populate and prepare the dataset required for digital twin ingestion.

In this configuration, the backend containers 3122 can interface with the digital twin service 3222 to perform simulation or emulation tasks using live or near-real-time operational data derived from the EMR system 3202. The integration allows the digital twin service 3222 to ingest operational data from the EMR system 3202 in real time, enabling scenario emulation, performance analysis, and operational optimization across connected hospital systems.

In various aspects, this configuration can support scenarios where customer LDAP 3106 is integrated with the IDAM 3110 and where customer data fabrics are configured to ingest EMR feeds. With appropriate customer authorization, the patient data store 3212 can share its output streams with the simulation and emulation platform without requiring additional integration effort from the customer, thereby enabling scalable, cloud-based operational emulation built atop existing clinical data infrastructure.

The export stream from the patient data store 3212 can flush EMR feeds to a streaming service, where a function such as the simulation event publisher 3218 can filter and transform the data. The transformed data can be published into the digital twin service 3222, either directly or through the data modeling service 3220. The digital twin service 3222 can then provide analysis on received data, which can be consumed directly by the customer or further enhanced by a provider system to generate additional insights prior to presentation to the customer.

In various aspects, the various embodiments described herein can comprise multiple software components (“services”) running on the Internet cloud. The Internet cloud can comprise a large number of virtual machines managed by a cloud provider. A virtual machine (e.g., virtual network 3102) can comprise an operating system running on a virtualization layer of a physical computer. A physical computer can comprise computer-readable memory and processors that can store and execute computer-executable (“software”) components.

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g.,support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z = (z₁, z₂, z₃, z₄, z n), to a confidence that the input belongs to a class, as by f(z) = confidence(class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

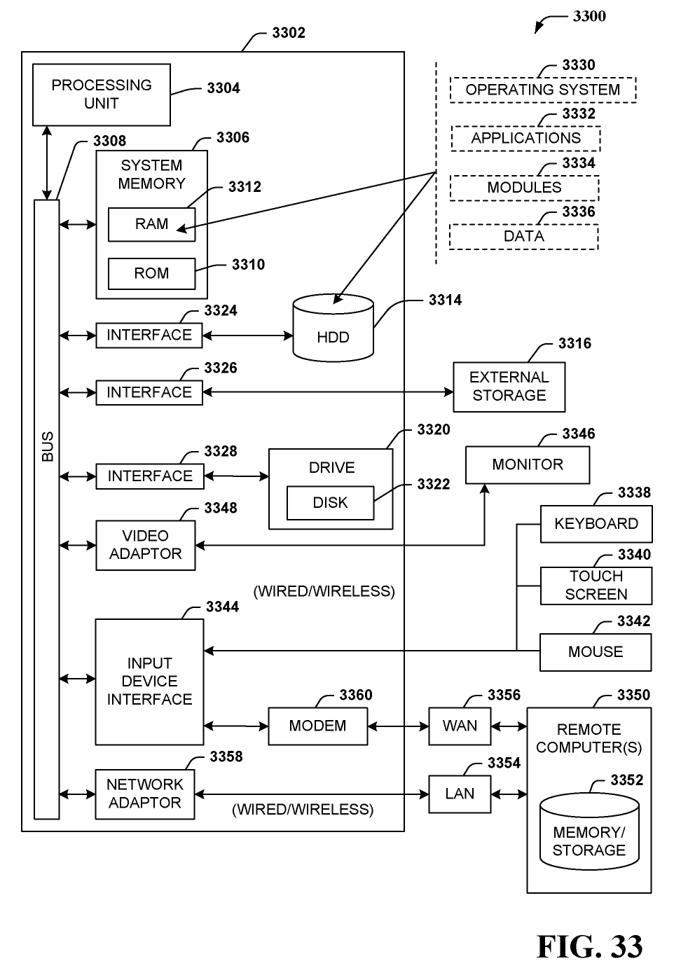

In order to provide additional context for various embodiments described herein, FIG. 33 and the following discussion are intended to provide a brief, general description of a suitable computing environment 3300 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 33, the example environment 3300 for implementing various embodiments of the aspects described herein includes a computer 3302, the computer 3302 including a processing unit 3304, a system memory 3306 and a system bus 3308. The system bus 3308 couples system components including, but not limited to, the system memory 3306 to the processing unit 3304. The processing unit 3304 can be any of various commercially available processors. Dual microprocessors and other multi-processor architectures can also be employed as the processing unit 3304.

The system bus 3308 can be any of several types of bus structure that can further interconnect to a memory bus (with or without a memory controller), a peripheral bus, and a local bus using any of a variety of commercially available bus architectures. The system memory 3306 includes ROM 3310 and RAM 3312. A basic input/output system (BIOS) can be stored in a non-volatile memory such as ROM, erasable programmable read only memory (EPROM), EEPROM, which BIOS contains the basic routines that help to transfer information between elements within the computer 3302, such as during startup. The RAM 3312 can also include a high-speed RAM such as static RAM for caching data.

The computer 3302 further includes an internal hard disk drive (HDD) 3314 (e.g., EIDE, SATA), one or more external storage devices 3316 (e.g., a magnetic floppy disk drive (FDD) 3316, a memory stick or flash drive reader, a memory card reader, etc.) and a drive 3320, e.g., such as a solid state drive, an optical disk drive, which can read or write from a disk 3322, such as a CD-ROM disc, a DVD, a BD, etc. Alternatively, where a solid state drive is involved, disk 3322 would not be included, unless separate. While the internal HDD 3314 is illustrated as located within the computer 3302, the internal HDD 3314 can also be configured for external use in a suitable chassis (not shown). Additionally, while not shown in environment 3300, a solid state drive (SSD) could be used in addition to, or in place of, an HDD 3314. The HDD 3314, external storage device(s) 3316 and drive 3320 can be connected to the system bus 3308 by an HDD interface 3324, an external storage interface 3326 and a drive interface 3328, respectively. The interface 3324 for external drive implementations can include at least one or both of Universal Serial Bus (USB) and Institute of Electrical and Electronics Engineers (IEEE) 3394 interface technologies. Other external drive connection technologies are within contemplation of the embodiments described herein.

The drives and their associated computer-readable storage media provide nonvolatile storage of data, data structures, computer-executable instructions, and so forth. For the computer 3302, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 3312, including an operating system 3330, one or more application programs 3332, other program modules 3334 and program data 3336. All or portions of the operating system, applications, modules, or data can also be cached in the RAM 3312. The systems and methods described herein can be implemented utilizing various commercially available operating systems or combinations of operating systems.

Computer 3302 can optionally comprise emulation technologies. For example, a hypervisor (not shown) or other intermediary can emulate a hardware environment for operating system 3330, and the emulated hardware can optionally be different from the hardware illustrated in FIG. 33. In such an embodiment, operating system 3330 can comprise one virtual machine (VM) of multiple VMs hosted at computer 3302. Furthermore, operating system 3330 can provide runtime environments, such as the Java runtime environment or the .NET framework, for applications 3332. Runtime environments are consistent execution environments that allow applications 3332 to run on any operating system that includes the runtime environment. Similarly, operating system 3330 can support containers, and applications 3332 can be in the form of containers, which are lightweight, standalone, executable packages of software that include, e.g., code, runtime, system tools, system libraries and settings for an application.

Further, computer 3302 can be enable with a security module, such as a trusted processing module (TPM). For instance with a TPM, boot components hash next in time boot components, and wait for a match of results to secured values, before loading a next boot component. This process can take place at any layer in the code execution stack of computer 3302, e.g., applied at the application execution level or at the operating system (OS) kernel level, thereby enabling security at any level of code execution.

A user can enter commands and information into the computer 3302 through one or more wired/wireless input devices, e.g., a keyboard 3338, a touch screen 3340, and a pointing device, such as a mouse 3342. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 3304 through an input device interface 3344 that can be coupled to the system bus 3308, but can be connected by other interfaces, such as a parallel port, an IEEE 3394 serial port, a game port, a USB port, an IR interface, a BLUETOOTH® interface, etc.

A monitor 3346 or other type of display device can be also connected to the system bus 3308 via an interface, such as a video adapter 3348. In addition to the monitor 3346, a computer typically includes other peripheral output devices (not shown), such as speakers, printers, etc.

The computer 3302 can operate in a networked environment using logical connections via wired or wireless communications to one or more remote computers, such as a remote computer(s) 3350. The remote computer(s) 3350 can be a workstation, a server computer, a router, a personal computer, portable computer, microprocessor-based entertainment appliance, a peer device or other common network node, and typically includes many or all of the elements described relative to the computer 3302, although, for purposes of brevity, only a memory/storage device 3352 is illustrated. The logical connections depicted include wired/wireless connectivity to a local area network (LAN) 3354 or larger networks, e.g., a wide area network (WAN) 3356. Such LAN and WAN networking environments are commonplace in offices and companies, and facilitate enterprise-wide computer networks, such as intranets, all of which can connect to a global communications network, e.g., the Internet.

When used in a LAN networking environment, the computer 3302 can be connected to the local network 3354 through a wired or wireless communication network interface or adapter 3358. The adapter 3358 can facilitate wired or wireless communication to the LAN 3354, which can also include a wireless access point (AP) disposed thereon for communicating with the adapter 3358 in a wireless mode.

When used in a WAN networking environment, the computer 3302 can include a modem 3360 or can be connected to a communications server on the WAN 3356 via other means for establishing communications over the WAN 3356, such as by way of the Internet. The modem 3360, which can be internal or external and a wired or wireless device, can be connected to the system bus 3308 via the input device interface 3344. In a networked environment, program modules depicted relative to the computer 3302 or portions thereof, can be stored in the remote memory/storage device 3352. It will be appreciated that the network connections shown are example and other means of establishing a communications link between the computers can be used.

When used in either a LAN or WAN networking environment, the computer 3302 can access cloud storage systems or other network-based storage systems in addition to, or in place of, external storage devices 3316 as described above, such as but not limited to a network virtual machine providing one or more aspects of storage or processing of information. Generally, a connection between the computer 3302 and a cloud storage system can be established over a LAN 3354 or WAN 3356 e.g., by the adapter 3358 or modem 3360, respectively. Upon connecting the computer 3302 to an associated cloud storage system, the external storage interface 3326 can, with the aid of the adapter 3358 or modem 3360, manage storage provided by the cloud storage system as it would other types of external storage. For instance, the external storage interface 3326 can be configured to provide access to cloud storage sources as if those sources were physically connected to the computer 3302.

The computer 3302 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

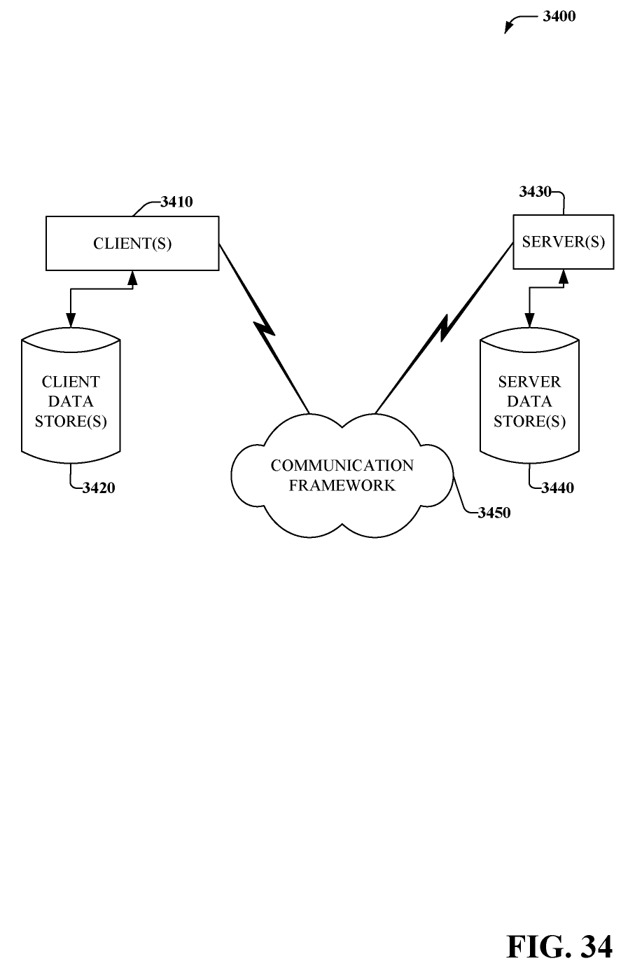

FIG. 34 is a schematic block diagram of a sample computing environment 3400 with which the disclosed subject matter can interact. The sample computing environment 3400 includes one or more client(s) 2330. The client(s) 2330 can be hardware or software (e.g., threads, processes, computing devices). The sample computing environment 3400 also includes one or more server(s) 3430. The server(s) 3430 can also be hardware or software (e.g., threads, processes, computing devices). The servers 3430 can house threads to perform transformations by employing one or more embodiments as described herein, for example. One possible communication between a client 2330 and a server 3430 can be in the form of a data packet adapted to be transmitted between two or more computer processes. The sample computing environment 3400 includes a communication framework 3450 that can be employed to facilitate communications between the client(s) 2330 and the server(s) 3430. The client(s) 2330 are operably connected to one or more client data store(s) 3420 that can be employed to store information local to the client(s) 2330. Similarly, the server(s) 3430 are operably connected to one or more server data store(s) 3440 that can be employed to store information local to the servers 3430.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the "C" programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For ease of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
at least one memory that stores computer executable components; and
at least one processor that executes the computer executable components stored in the at least one memory, wherein the computer executable components comprise:
a modeling component that:
generates scenario configuration data and scenario configuration metadata representing a configuration of resources or patient flow of one or more hospital units;
stores the scenario configuration data in object storage; and
stores the scenario configuration metadata in metadata storage;
a simulation component that:
retrieves the scenario configuration metadata from the metadata storage;
accesses the scenario configuration data based on the scenario configuration metadata;
simulates a scenario in the one or more hospital units using the scenario configuration data and the scenario configuration metadata representing the configuration within one or more containerized runtime instances to generate simulation data;
updates the scenario configuration data in the object storage based on the simulation data; and
updates metadata stored in the metadata storage to reflect updated scenario configuration data; and
an analysis component that:
dynamically invokes at least one large language model (LLM) to generate, within one or more containerized runtime instances, action plan recommendations for operational bottlenecks detected based on the simulation data; and
stores the action plan recommendations in the object storage, and
wherein the one or more containerized runtime instances execute within a layered virtualized computing environment that provides isolation between the one or more containerized runtime instances.

2. The system of claim 1, wherein the modeling component determines, via the at least one LLM, the configuration based on natural language input or machine-readable input.

3. The system of claim 1, wherein the modeling component detects gaps in the configuration and generates user prompts to fill in the gaps in the configuration.

4. The system of claim 2, wherein the modeling component translates, via a language embedding model, user input into machine-readable input.

5. The system of claim 1, wherein the analysis component stores the simulation data in a knowledge base for subsequent access or retrieval.

6. The system of claim 2, wherein the analysis component generates, via the at least one LLM, a summary from the simulation data.

7. The system of claim 1, wherein the analysis component recommends alternate configurations of the one or more hospital units, and wherein the simulation component simulates the scenario using the alternate configurations.

8. The system of claim 7, wherein the analysis component identifies changes in the simulation data between the simulations of the scenario with the configuration and the alternate configurations.

9. The system of claim 1, wherein the simulation data comprises at least one of: patient transitions, patient wait times, or resource utilization.

10. The system of claim 1, wherein the analysis component generates visual graphics based on the simulation data.

11. The system of claim 1, wherein the computer executable components further comprise:
a security component that manages data access controls, encrypts user data, or regulates system traffic.

12. The system of claim 1, wherein the modeling component models the patient flow based on at least one of: user input, historical electronic medical records (EMR), live EMR feeds, or artificial intelligence-generated patient flows.

13. The system of claim 1, wherein the simulation component simulates the scenario based on hospital data.

14. The system of claim 1, wherein the analysis component applies the action plan recommendations to operational configuration data associated with the one or more hospital units.

15. A computer-implemented method, comprising:
generating, by a system operatively coupled to a processor, a configuration of resources or patient flow of one or more hospital units;
simulating, by the system, a scenario in the one or more hospital units using the configuration to generate simulation data; and
generating, by the system, action plan recommendations for operational bottlenecks detected based on the simulation data.

16. The computer-implemented method of claim 15, further comprising:
detecting, by the system, gaps in the configuration; and
generating, by the system, user prompts to fill in the gaps in the configuration.

17. The computer-implemented method of claim 15, further comprising:
determining, by the system, via a large language model (LLM), the configuration based on natural language input or machine-readable input.

18. The computer-implemented method of claim 17, further comprising:
generating, by the system, via the LLM, a summary or visual graphics from the simulation data.

19. The computer-implemented method of claim 15, further comprising:
recommending, by the system, alternate configurations of the one or more hospital units; and
simulating, by the system, the scenario using the alternate configurations; and
identifying, by the system, changes in the simulation data between the simulations of the scenario with the configuration and the alternate configurations.

20. A computer program product for optimizing hospital operations, the computer program product comprising a computer readable storage medium having program instructions embodied therewith, the program instructions executable by a processor to cause the processor to:
generate, by the processor, a configuration of resources or patient flow of one or more hospital units;
simulate, by the processor, a scenario in the one or more hospital units using the configuration to generate simulation data; and
generate, by the processor, action plan recommendations for operational bottlenecks detected based on the simulation data.
